(19) United States
(12) Patent Application Publication
(10) Pub. No.: US 2009/0131420 A1
(43) Pub. Date: May 21, 2009

(54) Novel Heterocyclic Analogs of Biphenyl Ethers

(76) Inventors: Surendrakumar S. Pandey, Tamil Nadu (IN); Gajendra Singh, Tamil Nadu (IN); Santhanagopalan Chithra, Tamil Nadu (IN); Gaddam O. Reddy, Tamil Nadu (IN); Somasundaram Sangeetha, Tamil Nadu (IN); Debendranath Dey, Tamil Nadu (IN)

Correspondence Address: OLIFF & BERRIDGE, PLC, P.O. BOX 320850, ALEXANDRIA, VA, 22320-4850, US

(21) Appl. No.: 11/992,951

(22) PCT Filed: Sep. 27, 2006
(86) PCT No.: PCT/IB2006/002676

§ 371 Date: Jun. 9, 2008

(30) Foreign Application Priority Data
Oct. 7, 2005 (IN) 1431/CHE/2005

(51) Int. Cl. A61K 31/5377; C07D 413/10; A61P 37/02; A61P 3/10

(52) U.S. Cl. 514/230.8; 544/131

(57) ABSTRACT

The present invention relates to novel compounds of formula (I) and their pharmaceutically acceptable salts and compositions. The present invention more particularly provides novel heterocyclic analogs of biphenyl ethers of the general formula (I).

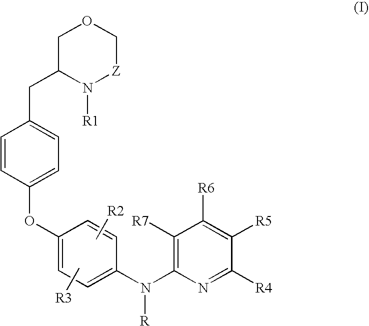

### FIELD OF THE INVENTION

The present invention relates to novel compounds of formula (I) and their pharmaceutically acceptable salts and compositions. The present invention more particularly provides novel heterocyclic analogs of biphenyl ethers of the general formula (I).

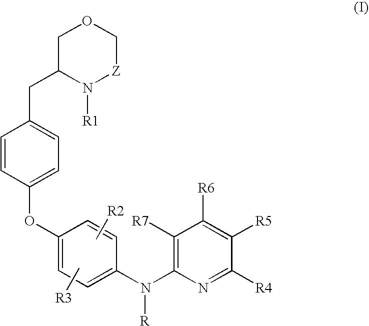

The present invention also relates to a process for the preparation of the above said novel compounds and their pharmaceutically acceptable salts and compositions. The compounds of the present invention are effective in lowering blood glucose, serum insulin, free fatty acids, cholesterol and triglyceride levels and are useful in the treatment and/or prophylaxis of type II diabetes. The compounds of the present invention are effective in the treatment of obesity, inflammation, autoimmune diseases such as multiple sclerosis and rheumatoid arthritis. Surprisingly, these compounds increase the leptin level and have no liver toxicity.

Furthermore, the compounds of the present invention are useful for the treatment of disorders associated with insulin resistance, such as polycystic ovary syndrome, as well as hyperlipidemia, coronary artery disease and peripheral vascular disease, and for the treatment of inflammation and immunological diseases, particularly those mediated by cytokines such as TNF-α, IL-1, IL-6, IL-1β and cyclooxygenases such as COX-2.

### BACKGROUND OF THE INVENTION

The causes of type I and II diabetes are not yet clear, although both genetics and environment seem to be the factors. Type I diabetes is an autonomic immune disease and the patient must take insulin to survive. Type II diabetes is a more common form and is a metabolic disorder resulting from the body's inability to make a sufficient amount of insulin or to properly use the insulin that is produced. Insulin secretion and insulin resistance are considered the major defects, however, the precise genetic factors involved in the mechanism remain unknown.

Patients with diabetes usually have one or more of the following defects:

Less production of insulin by the pancreas;

Over secretion of glucose by the liver;

Independence of the glucose uptake by the skeletal muscles;

Defects in glucose transporters, desensitization of insulin receptors; and

Defects in the metabolic breakdown of polysaccharides.

Other than the parenteral or subcutaneous administration of insulin, there are about four classes of oral hypoglycemic agents used i.e. sulfonylureas, biguanides, alpha glucosidase inhibitors and thiazolidinediones.

Each of the current agents available for use in the treatment of diabetes has certain disadvantages. Accordingly, there is a continuing interest in the identification and development of new agents, which can be orally administered, for use in the treatment of diabetes.

The thiazolidinedione class listed above has gained more widespread use in recent years for the treatment of type II diabetes, exhibiting particular usefulness as insulin sensitizers to combat “insulin resistance”, a condition in which the patient becomes less responsive to the effects of insulin. There is a continuing need for non-toxic and more widely effective insulin sensitizers. With this objective in mind we have prepared the above-mentioned novel compounds.

With an objective of providing compounds, which are effective for such treatments as well as for the treatment of, for example, insulin resistance, hyperlipidemia, obesity, inflammation, multiple sclerosis and arthritis, we have continued our research to develop new thiazoldinediones along with other heterocyclic analogs.

Few Prior Art Reference which Disclose the Closest Compounds are Given Here:
i) International publication No. WO 01/02377 discloses compounds of formula (IIa), as telomerase inhibitors,

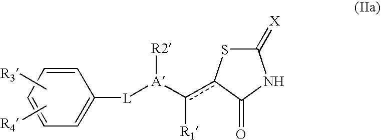

wherein R′₁ and R′₂ represents hydrogen, alkyl etc.; X represents oxygen or sulfur; ---- is a single or double bond; L represents oxygen, nitrogen, sulfur; R′₃ represents hydrogen, alkyl, aryl etc.; R′₄ represents hydrogen, alkyl, aryl etc.; A′ represents aryl.

An example of these compounds is shown in formula (IIb)

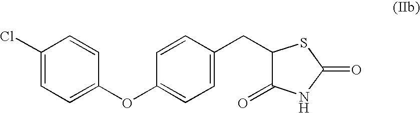

ii) EP 1148054 discloses compounds of formula (IIc)

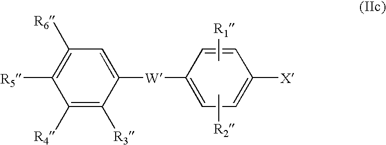

wherein R₁″, R₂″, R₃″, R₅″, R₆″, represent hydrogen, alkyl etc.; X′ represents methylene thiazolidin-2,4-dione, methylene oxazolidin-2,4-dione etc.; W′ represents oxygen, sulfur; R₄″ represents hydrogen, alkyl substituted with 0 to 3, substituents etc.

An example of these compounds is shown in formula (IId)

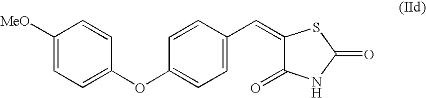

iii) U.S. Pat. No. 6,331,633 discloses compounds of formula (IIe)

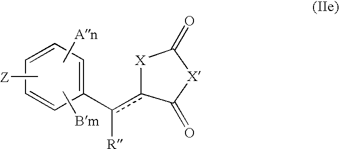

wherein Z is

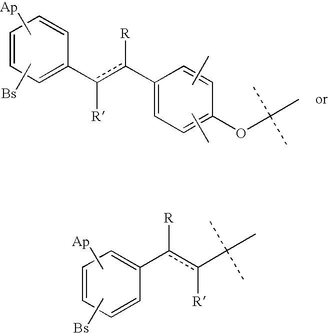

wherein n, m, q and r are independently integers from 0 to 4; p and s are independently integers from 0 to 5; a, b and c are double bonds which may be present or absent; R, R′ and R″ are independently H, C₁-C₂₀ linear or branched alkyl, C₂-C₂₀ linear or branched alkenyl, —CO₂H, —CO₂R′″, —NH₂, —NHR′″, —NR₂′″, —OH, —OR′″, halo, substituted C₁-C₂₀ linear or branched alkyl or substituted C₂-C₂₀ linear or branched alkenyl, wherein R′″ is C₁-C₂₀ linear or branched alkyl or linear or branched alkenyl; A, A′ and A″ are independently H, C₁-C₂₀ acylamino, C₁-C₂₀acyloxy, C₁-C₂₀alkanoyl, C₁-C₂₀ alkoxycarbonyl, C₁-C₂₀alkoxy, C₁-C₂₀alkylamino, C₁-C₂₀alkylcarboxylamino, carboxyl, cyano, halo, hydroxy; B, B′ and B″ are independently H, C₁-C₂₀acylamino, C₁-C₂₀ acyloxy, C₁-C₂₀ alkanoyl, etc.; X, X′ are independently —NH, —NR′″, O or S.

An example of these compounds is shown in formula (IIf)

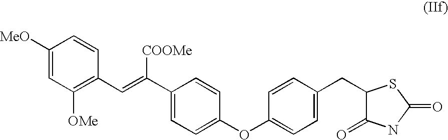

iv) U.S. Pat. No. 5,232,925 discloses compounds of formula (I)

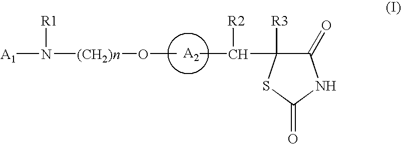

Compounds of formula (I) or a tautomeric form thereof, or a pharmaceutically acceptable salt thereof, or a pharmaceutically acceptable solvate thereof, wherein: A¹ represents a substituted or unsubstituted aromatic heterocyclyl group; R¹ represents a hydrogen atom, an alkyl group, an acyl group, an aralkyl group, wherein the aryl moiety may be substituted or unsubstituted, or a substituted or unsubstituted aryl group; R² and R³ each represent hydrogen, or R² and R³ together represent a bond; A² represents a benzene ring having in total up to five substituents; and n represents an integer in the range of from 2 to 6; pharmaceutical compositions containing such compounds and the use of such compounds and compositions in medicine

### OBJECTIVE OF THE INVENTION

With an objective to develop novel compounds for lowering blood glucose, free fatty acids, cholesterol and triglyceride levels in type II diabetes and to treat autoimmune diseases such as multiple sclerosis and rheumatoid arthritis, we focused our research to develop new compounds effective in the treatment of the above-mentioned diseases. Efforts in this direction have led to compounds having the general formula (I).

The main objective of the present invention is therefore, to provide novel compounds of the general formula (I), and their pharmaceutically acceptable salts and compositions.

Another objective of the present invention is to provide novel compounds of the general formula (I), and their pharmaceutically acceptable salt and compositions that are useful for treatment of disorders associated with insulin resistance, such as polycystic ovary syndrome, as well as hyperlipidemia, coronary artery disease, peripheral vascular disease, and for the treatment of inflammation and immunological diseases, particularly those mediated by cytokines such as TNF-α, IL-1, IL-6, IL-1β and cyclooxygenases such as COX-2.

Another objective of the present invention is to provide novel compounds of the general formula (I), and their pharmaceutically acceptable salts and compositions having enhanced activities, without toxic effects or with reduced toxic effects.

Yet another objective of the present invention is to provide a process for the preparation of novel compounds of the general formula (I), and their pharmaceutically acceptable salts and compositions.

### SUMMARY OF THE INVENTION

The present invention relates to novel compounds of the general formula (I),

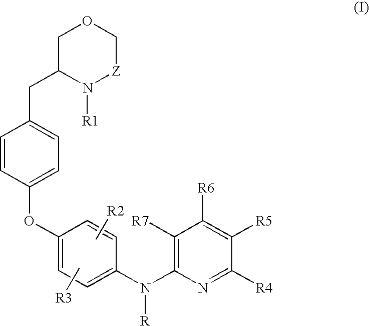

their pharmaceutically acceptable salts and compositions; wherein Z represents CH₂, C═O; wherein R and R₁ may be same or different and independently represent hydrogen, substituted or unsubstituted groups selected from alkyl, alkenyl, aryl, aryloxy, alkoxy, COR₈, wherein R₈ represents substituted or unsubstituted groups selected from alkyl, alkenyl, aryl, aryloxy, alkoxy, heteroaryl or heterocyclyl; R₂ and R₃, may be same or different and independently represent hydrogen, halogen, hydroxy, nitro, cyano, formyl, amino, alkyl, haloalkyl, alkoxy group; R₄, R₅, R₆ and R₇ may be same or different and independently represents hydrogen, nitro, cyano, formyl, azido, halo, or substituted or unsubstituted groups selected from alkyl, alkoxy, acyl, haloalkyl, amino, hydrazine, monoalkylamino, dialkylamino, acylamino, alkylsulfonyl, alkylsulfinyl, arylsulfonyl, arylsulfinyl, alkylthio, arylthio, alkoxycarbonyl, aryloxycarbonyl, alkoxyalkyl, sulfamoyl, carboxylic acid or its derivatives.

### DETAILED DESCRIPTION OF THE INVENTION

Z represents CH₂, C═O; suitable groups represented by R and R₁ may be same or different and independently represent hydrogen, alkyl, alkenyl, substituted or unsubstituted groups selected from (C₁-C₄) alkyl groups such as methyl, ethyl, propyl, isopropyl, n-butyl, isobutyl, t-butyl and the like; substituted or unsubstituted linear or branched (C₂-C₇) alkenyl groups such as ethenyl, propenyl, butenyl and the like; aryl groups such as phenyl, naphthyl and the like, the aryl group may be substituted; aryloxy, substituted or unsubstituted linear or branched (C₂-C₅) alkoxy groups such as methoxy, ethoxy, propoxy, n-butoxy, isobutoxy, t-butoxy and the like; COR₈, where R₈ represents substituted or unsubstituted groups selected from (C₁-C₄) alkyl groups such as methyl, ethyl, propyl, isopropyl, n-butyl, isobutyl, t-butyl and the like; substituted or unsubstituted linear or branched (C₂-C₅) alkenyl groups such as ethenyl, propenyl, butenyl and the like; aryl groups such as phenyl, naphthyl and the like, the aryl group may be substituted; aryloxy, substituted or unsubstituted linear or branched (C₂-C₅) alkoxy groups such as methoxy, ethoxy, propoxy, n-butoxy, isobutoxy, t-butoxy and the like.

Suitable groups represented by R₂ and R₃ are selected from hydrogen, halogen atoms such as fluorine, chlorine, bromine or iodine; hydroxy, nitro, cyano, formyl, amino, unsubstituted linear or branched (C₁-C₄) alkyl groups such as methyl, ethyl, n-propyl, isopropyl, n-butyl, isobutyl, t-butyl and the like; haloalkyl groups such as chloromethyl, chloroethyl, trifluoromethyl, trifluoroethyl, dichloromethyl, dichloroethyl, trichloromethyl, difluoromethyl, and the like, which may be substituted; alkoxy groups such as methoxy, ethoxy, n-propoxy, isopropoxy and the like, which may be substituted.

R₄, R₅, R₆ and R₇ may be same or different and independently represent hydrogen, halogen atoms such as fluorine, chlorine, bromine or iodine; hydroxy, nitro, cyano, formyl, amino, azido, hydrazine; unsubstituted or unsubstituted groups selected from linear or branched (C₁-C₄) alkyl groups such as methyl, ethyl, n-propyl, isopropyl, n-butyl, isobutyl, t-butyl and the like; haloalkyl groups such as chloromethyl, chloroethyl, trifluoromethyl, trifluoroethyl, dichloromethyl, dichloroethyl, trichloromethyl, difluoromethyl, and the like, which may be substituted; alkoxy groups such as methoxy, ethoxy, n-propoxy, isopropoxy and the like, which may be substituted; monoalkylamino groups such as —NHCH₃, —NHC₂H₅, —NHC₃H₇, —NHC₆H₁₃, and the like, which may be substituted; dialkylamino groups such as —N(CH₃)₂, —NCH₃(C₂H₅), —N(C₂H₅)₂ and the like, which may be substituted; carboxylic acids or its derivatives such as esters or amides; acylamino groups such as —NHC(—O)CH₃, —NHC(—O)C₂H₅, —NHC(═O)C₃H₇, —NHC(═O)C₆H₁₃, and the like, which may be substituted; alkylsulfonyl groups such as methylsulfonyl, ethylsulfonyl, n-propylsulfonyl, iso-propylsulfonyl and the like, the alkylsulfonyl group may be substituted; arylsulfonyl groups such as phenylsulfonyl or naphthylsulfonyl, the arylsulfonyl group may be substituted; alkylsulfinyl groups such as methylsulfinyl, ethylsulfinyl, n-propylsulfinyl, iso-propylsulfinyl and the like, the alkylsulfinyl group may be substituted; arylsulfinyl groups such as phenylsulfinyl or naphthylsulfinyl, the arylsulfinyl group may be substituted; alkylthio groups such as methylthio, ethylthio, n-propylthio, iso-propylthio and the like, the alkylthio group may be substituted; alkoxycarbonyl groups such as methoxycarbonyl, ethoxycarbonyl, n-propoxycarbonyl, isopropoxycarbonyl and the like, the alkoxycarbonyl group may be substituted; aryloxycarbonyl groups such as phenoxycarbonyl, napthoxycarbonyl, and the like, the aryloxycarbonyl group may be substituted; alkoxyalkyl groups such as methoxymethyl, ethoxymethyl, methoxyethyl, ethoxyethyl and the like, which may be substituted; sulfamoyl; carboxylic acid or its derivatives.

Suitable substituents on the groups represented by R₁, R₂, R₃ R₄, R₅, R₆, R₇, and R₈ are selected from nitro, cyano, hydroxy, halo, formyl, azido, alkyl, alkoxy, acyl, haloalkyl, amino, hydrazine, monoalkylamino, dialkylamino, acylamino, alkylsulfonyl, alkylsulfinyl, arylsulfonyl, arylsulfinyl, alkylthio, arylthio, alkoxycarbonyl, aryloxycarbonyl, alkoxyalkyl, sulfamoyl, carboxylic acid or its derivatives.

Pharmaceutically acceptable salts of the present invention include alkali metals like Li, Na, and K, alkaline earth metals like Ca and Mg, salts of organic bases such as diethanolamine, α-phenylethylamine, benzylamine, piperidine, morpholine, pyridine, hydroxyethylpyrrolidine, hydroxyethylpiperidine, choline and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate, which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprise other solvents of crystallization such as alcohols.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. The compositions may be prepared by processes known in the art. The amount of the active ingredient in the composition may be less than 60% by weight. Such compositions typically contain from 1 to 25%, preferably 1 to 15% by weight of active compound, the remainder of the composition being pharmaceutically acceptable carriers, diluents, excipients or solvents.

The protecting groups used in the invention are conventional protecting groups such as t-butoxycarbonyl (t-Boc), trityl, trifluoroacetyl, benzyloxy, benzyloxy carbonyl (Cbz) and the like. Deprotection can be done by conventional methods.

### Particularly Useful Compounds According to the Present Invention Include:

1). 5-{4-[4-(Pyridin-2-ylamino)phenoxy]benzyl}morpholin-3-one;
2). 5-(4-{4-[(5-Nitropyridin-2-yl)amino]phenoxy}benzyl)morpholin-3-one;
3). 5-(4-{4-[5-(Trifluoromethyl)pyridin-2-yl)amino]phenoxy}benzyl)morpholin-3-one;
4). 4-Methyl-5-(4-{4-[(5-nitropyridin-2-yl)amino]phenoxy}benzyl)morpholin-3-one;
5). 5-(4-{4-[(3-Nitropyridin-2-yl)amino]phenoxy}benzyl)morpholin-3-one;
6). 5-{4-[2-Fluoro-4-(3-nitropyridin-2-ylamino)phenoxy]benzyl}morpholin-3-one;
7). 2-[(4-{4-[(5-Oxomorpholin-3-yl)methyl]phenoxy}phenyl)amino]nicotinonitrile;
8). 5-{4-[2-Fluoro-4-(5-nitropyridin-2-ylamino)phenoxy]benzyl}morpholin-3-one;
9). 5-{4-[2-Fluoro-4-(5-(trifluoromethyl)pyridin-2-ylamino)phenoxy]benzyl}morpholin-3-one;
10). 2-[(3-Fluoro-4-{4-[(5-oxomorpholin-3-yl)methyl]phenoxy}phenyl)amino]nicotinonitrile;
11). 4-Methyl-5-{4-[2-fluoro-4-(3-nitropyridin-2-ylamino)phenoxy]benzyl}morpholin-3-one;
12). 4-Methyl-5-{4-[4-(5-nitropyridin-2-ylamino)phenoxy]benzyl}morpholin-3-one;
13). 4-Methyl-5-(4-{4-[5-(trifluoromethyl)pyridin-2-yl)amino]phenoxy}benzyl)morpholin-3-one;
14). 5-(4-{4-[Methyl(pyridin-2-yl)amino]phenoxy}benzyl)-4-methyl morpholin-3-one;
15). 4-Methyl-5-{4-[2-fluoro-4-(5-nitropyridin-2-ylamino)phenoxy]benzyl}morpholin-3-one;
16). 4-Methyl-5-{4-[2-fluoro-4-(5-(trifluoromethyl)pyridin-2-ylamino)phenoxy]benzyl}morpholin-3-one;
17). 2-[(3-Fluoro-4-{4-[(4-methyl-5-oxomorpholin-3-yl)methyl]phenoxy}phenyl)amino]nicotinonitrile;
18). 2-[(4-{4-[(4-Methyl-5-oxomorpholin-3-yl)methyl]phenoxy}phenyl)amino]nicotinonitrile;
19). 4-Methyl-5-{4-[2-fluoro-4-(pyridin-2-ylamino)phenoxy]benzyl}morpholin-3-one;
20). 5-(4-{2-Fluoro-4-[methyl(5-nitropyridin-2-yl)amino]phenoxy}benzyl)-4-methyl morpholin-3-one;
21). 5-(4-{2-Fluoro-4-[methyl(3-nitropyridin-2-yl)amino]phenoxy}benzyl)-4-methyl morpholin-3-one;
22). 5-(4-{4-[Ethyl(5-nitropyridin-2-yl)amino]phenoxy}benzyl)-4-methyl morpholin-3-one;
23). 5-(4-{2-Fluoro-4-[ethyl(5-nitropyridin-2-yl)amino]phenoxy}benzyl)-4-methyl morpholin-3-one;
24). 5-(4-{4-[Methyl(3-nitropyridin-2-yl)amino]phenoxy}benzyl)-4-methyl morpholin-3-one;
25). 5-(4-{2-Fluoro-4-[ethyl(5-(trifluoromethyl)pyridin-2-yl)amino]phenoxy}benzyl)-4-methyl morpholin-3-one;
26). 5-(4-{4-[Ethyl(pyridin-2-yl)amino]phenoxy}benzyl)-4-methyl morpholin-3-one;
27). 5-(4-{4-[Methyl(5-nitropyridin-2-yl)amino]phenoxy}benzyl)-4-methyl morpholin-3-one;
28). 5-{4-[4-(5-Nitropyridin-2-ylamino)phenoxy]benzyl}-4-ethyl morpholin-3-one;
29). 2-[Methyl(4-{4-[(4-methyl-5-oxomorpholin-3-yl)methyl]phenoxy}phenyl)amino]nicotinonitrile;
30). -5-{4-[2-Fluoro-4-(3-nitropyridin-2-ylamino)phenoxy]benzyl}-4-ethyl morpholin-3-one;
31). 2-[(3-Fluoro-4-{4-[(4-methyl-5-oxomorpholin-3-yl)methyl]phenoxy}phenyl)(methyl)amino]nicotinonitrile;
32). 5-{4-[4-(3-Nitropyridin-2-ylamino)phenoxy]benzyl}-4-ethyl morpholin-3-one;
33). 5-{4-[2-Fluoro-4-(pyridin-2-ylamino)phenoxy]benzyl}-4-ethyl morpholin-3-one;
34). 2-[(4-{4-[(4-Ethyl-5-oxomorpholin-3-yl)methyl]phenoxy}phenyl)amino]nicotinonitrile;
35). 5-{4-[4-(Pyridin-2-ylamino)phenoxy]benzyl}-4-ethyl morpholin-3-one;
36). 5-{4-[2-Fluoro-4-(5-nitropyridin-2-ylamino)phenoxy]benzyl}-4-ethyl morpholin-3-one;
37). 2-[(3-Fluoro-4-{4-[(4-ethyl-5-oxomorpholin-3-yl)methyl]phenoxy}phenyl)amino]nicotinonitrile;
38). N-Ethyl-3-nitro-N-{4-[4-{(4-methylmorpholin-3-yl)methyl}phenoxy]phenyl}-pyridin-2-amine;
39). 2-[(4-{4-[(4-Ethyl-5-oxomorpholin-3-yl)methyl]phenoxy}phenyl)amino]nicotinonitrile;
40). 5-(4-{2-Fluoro-4-[(5-(trifluoromethyl)pyridin-2-yl)amino]phenoxy}benzyl)-4-ethyl morpholin-3-one;
41). 5-(4-{2-Fluoro-4-[methyl(3-nitropyridin-2-yl)amino]phenoxy}benzyl)-4-ethyl morpholin-3-one;
42). 5-(4-{2-Fluoro-4-[methyl(5-nitropyridin-2-yl)amino]phenoxy}benzyl)-4-ethyl morpholin-3-one;
43). 5-(4-{2-Fluoro-4-[ethyl(5-nitropyridin-2-yl)amino]phenoxy}benzyl)-4-ethyl morpholin-3-one;
44). 5-(4-{4-[(5-(Trifluoromethyl)pyridin-2-yl)amino]phenoxy}benzyl)-4-ethyl morpholin-3-one;
45). 2-[(4-{4-[(4-Ethyl-5-oxomorpholin-3-yl)methyl]phenoxy}phenyl)(ethyl)amino]nicotinonitrile;
46). 2-[(3-Fluoro-4-{4-[(4-ethyl-5-oxomorpholin-3-yl)methyl]phenoxy}phenyl)(methyl)amino]nicotinonitrile;
47). 2-[(3-Fluoro-4-{4-[(4-ethyl-5-oxomorpholin-3-yl)methyl]phenoxy}phenyl)(ethyl)amino]nicotinonitrile;
48). 5-(4-{2-Fluoro-4-[ethyl(5-(trifluoromethyl)pyridin-2-yl)amino]phenoxy}benzyl)-4-ethyl morpholin-3-one;
49). 5-(4-{2-Fluoro-4-[methyl(pyridin-2-yl)amino]phenoxy}benzyl)-4-ethyl morpholin-3-one;
50). 5-(4-{4-[Methyl(5-(trifluoromethyl)pyridin-2-yl)amino]phenoxy}benzyl)-4-ethyl morpholin-3-one;
51). 5-(4-{4-[Methyl(3-nitropyridin-2-yl)amino]phenoxy}benzyl)-4-ethyl morpholin-3-one;
52). 5-(4-{4-[ethyl(3-nitropyridin-2-yl)amino]phenoxy}benzyl)-4-ethyl morpholin-3-one;
53). 5-(4-{4-[Methyl(5-(trifluoromethyl)pyridin-2-yl)amino]phenoxy}benzyl)-4-methyl morpholin-3-one;
54). 5-(4-{4-[Ethyl(5-(trifluoromethyl)pyridin-2-yl)amino]phenoxy}benzyl)-4-methyl morpholin-3-one;
55). 5-Nitro N-{4-[4-{(4-methylmorpholin-3-yl)methyl}phenoxy]phenyl}-pyridin-2-amine;
56). N-Ethyl-5-nitro-N-{4-[4-{(4-methylmorpholin-3-yl)methyl}phenoxy]phenyl}-pyridin-2-amine;
57). 3-Nitro-N-{4-[4-{(4-methylmorpholin-3-yl)methyl}phenoxy]phenyl}-pyridin-2-amine;

### Scheme:

a) Deprotection of the compound of formula (1a), gave the compound of formula (2a), which was further acylated to give the compound of formula (3a). The compound of formula (1a) is prepared according to the procedure described in Tetrahedron Asymmetry 14, 2003.

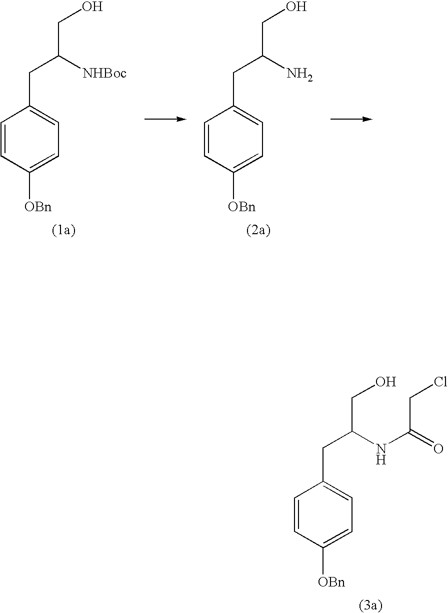

b) Cyclization of the compound of formula (3a) gave the compound of formula (4a), which was optionally further alkylated to give the compound of formula (5a), wherein all the other symbols are as defined earlier.

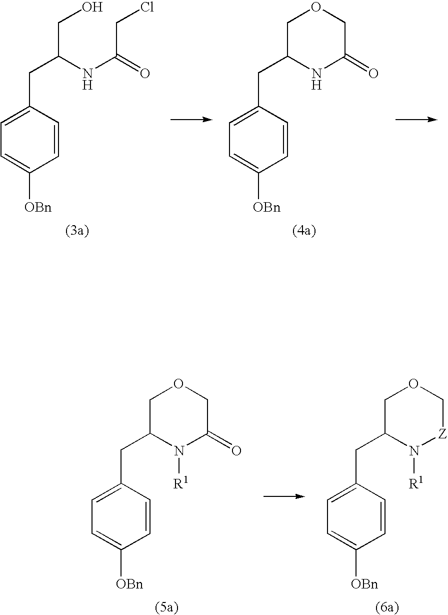

c) Optional reduction of the compound of formula (5a) gave the compound of formula (6a), wherein all the symbols are as defined earlier.

d) Debenzylation of compound of formula (6a) gave the compound of formula (7a), wherein all the symbols are as defined earlier.

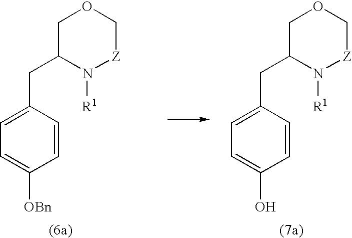

e) Reaction of the compound of formula (7a) with a compound of formula (8a), gave the compound of formula (9a), wherein all the symbols are as defined earlier.

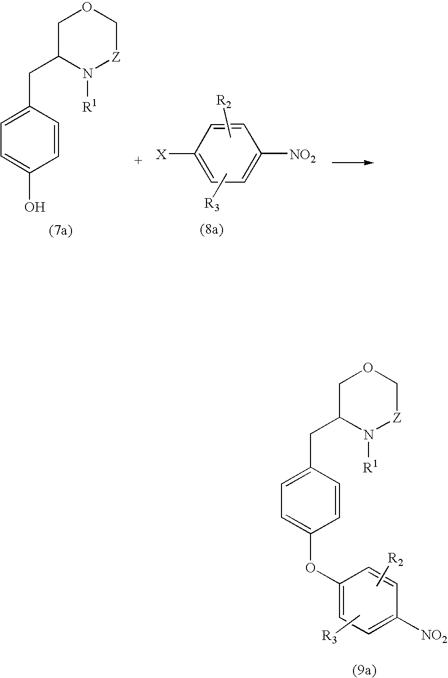

f) Reduction of the compound of formula (9a) gave the compound of formula (10a), and further condensation of the compound of formula (10a) with the compound of formula (11a) gave the compound of formula (I), wherein X represents halogen, Z represents —CH₂ or —C═O and all the other groups are as defined earlier.

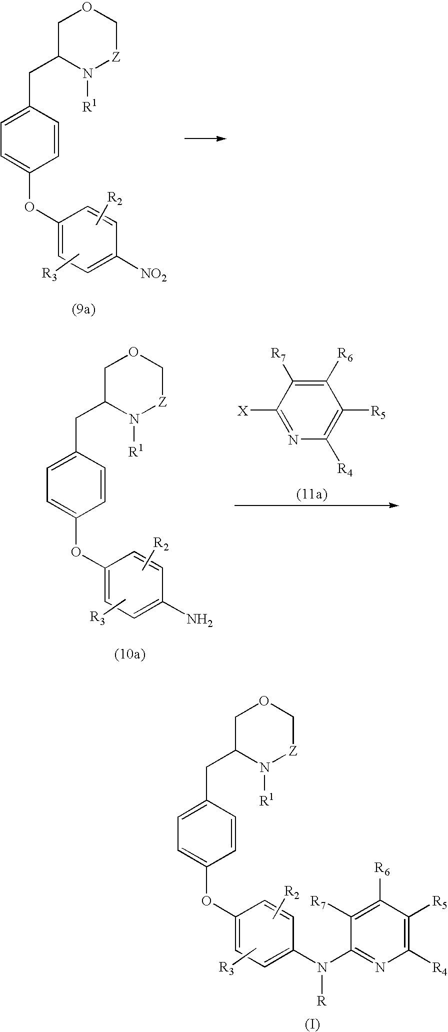

### The Reactions Described in the Processes Outlined Above are Performed by Using the Methods Described Herein

Deprotection of the compound of formula (1a) to the compound of formula (2a) may be carried out by using acids such as HCl, sulfuric acid and acetic acid in the presence of solvents such as dichloromethane, ethyl acetate, water and the like or a mixture thereof at a temperature in the range of −10° C. to 50° C.

The reaction of the compound of formula (2a) with chloro acetyl chloride is carried out in the presence of solvents such as dichloromethane, tetrahydrofuran, dimethylformamide, dimethyl sulfoxide, DME and the like or a mixture of solvents may be used to produce the compound of formula (3a). The reaction may be carried out in an inert atmosphere and may be effected in the presence of a base such as triethylamine, K₂CO₃, Na₂CO₃, NaH or mixtures thereof. The reaction temperature may range from 0° C. to 50° C., preferably at a temperature in the range of 0° C. to 10° C. The duration of the reaction may range from 1 to 12 hours, preferably from 2 to 6 hours.

Cyclisation of the compound of formula (3a) is carried out in the presence of a base such as potassium t-.butoxide, NaH and in the presence of a solvent such as t-butanol, isopropanol, toluene, methoxyethanol or mixtures thereof to yield a compound of formula (4a). The reaction temperature may range from 0° C. to 50° C., preferably at a temperature in the range of 10° C. to 40° C. The duration of the reaction may range from 1 to 12 hours, preferably from 2 to 6 hours.

The reaction of formula (4a) with alkyl halide is carried out in the presence of solvents such as dichloromethane, tetrahydrofuran, dimethylformamide, dimethyl sulfoxide, DME and the like or a mixture of solvents may be used to produce the compound of formula (5a). The reaction may be carried out in an inert atmosphere and may be effected in the presence of a base such as triethylamine, K₂CO₃, Na₂CO₃, NaH or mixtures thereof. The reaction temperature may range from 0° C. to 50° C., preferably at a temperature in the range of 0° C. to 10° C. The duration of the reaction may range from 1 to 12 hours, preferably from 2 to 6 hours.

Reduction of the compound of formula (5a) to the compound of formula (6a) may be carried out in the presence of a catalyst such as sodium borohydride (NaBH₄), Lithium Aluminum Hydride (LiH), zinc-mercury amalgam, hydrazine and the like. The reaction may be conducted in the presence of solvents such as methanol, dichloromethane, dioxane, acetic acid, ethyl acetate and the like or mixtures thereof.

Debenzylation of the compound of formula (6a) to the compound of formula (7a) may be carried out in the presence of H₂/catalyst. The catalyst may be selected from Pd/C, Rh/C, Pt/C, Raney Nickel, and the like or a mixture of catalysts may be used. The reaction may be conducted in the presence of solvents such as methanol, dichloromethane, dioxane, acetic acid, ethyl acetate and the like or mixtures thereof. A pressure between atmospheric pressure to 100 psi may be employed. The catalyst may be 5-10% Pd/C and the amount of catalyst used may range from 50-300% w/w.

The reaction of the compound of formula (7a) with the compound of formula (8a), is carried out in the presence of solvents such as tetrahydrofuran, dimethylformamide, dimethyl sulfoxide, DME and the like or a mixture of solvents thereof, may be used to produce the compound of formula (9a). The reaction may be carried out in an inert atmosphere and may be effected in the presence of a base such as K₂CO₃, Na₂CO₃, NaH or mixtures thereof. The reaction temperature may range from 60° C. to 150° C., preferably at a temperature in the range of 80° C. to 100° C. The duration of the reaction may range from 1 to 24 hours, preferably from 2 to 6 hours.

Hydrogenation of the compound of the formula (9a) is carried out by using catalysts such as Raney nickel, Pd/C, in the presence of solvents such as, methanol, ethanol, ethylacetate, n-butylacetate or a mixture thereof. The reaction may be carried out at 0° C. to 50° C. The duration of the reaction may range from 2 to 24 hours, to produce a compound of formula (10a).

The compound of formula (10a) is reacted with compound of formula (11a) in the presence of solvents such as toluene, methanol, ethanol, tetrahydrofuran, chloroform, dichloromethane, dichloroethane, ethylacetate, o-dichlorobenzene or a mixture thereof. The reaction may be carried out at 50° C. to 150° C. The duration of the reaction may range from 2 to 24 hours, to produce a compound of formula (I). The invention is explained in detail in the examples given below which are provided by way of illustration only and therefore should not be construed to limit the scope of the invention.

### EXAMPLE 1

### Synthesis of 4-{4-[4-(Pyridin-2-ylamino)phenoxy]benzyl}-morpholin-3-one

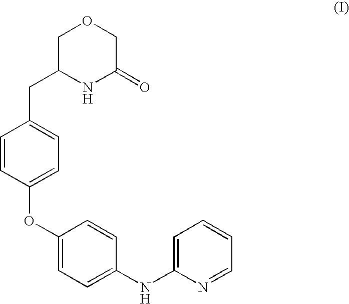

### Step I

### Synthesis of 2-Amino-3-[4-(benzyloxy)phenyl]propan-1-ol Hydrochloride

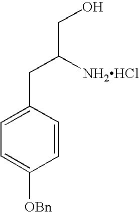

Dry HCl gas was bubbled into a, solution of t-butyl-2-hydroxy-1-(4-benzyloxybenzyl)ethylcarbamate (4 g, 11.20 mmol) in dichloromethane (50 ml) at 0-5° C. for two hours. After completion of reaction, excess of the HCl gas was removed by bubbling nitrogen gas. The white solid thus obtained was filtered and dried to yield 2-amino-3-[4-(benzyloxy)phenyl]propan-1-ol hydrochloride (2.35 g). ¹HNMR (CDCl₃ 400 MHz): δ 2.46 (q, 1H), 2.73 (dd, 1H), 3.06 (m, 1H), 3.35 (m, 1H), 3.63 (dd, 1H) 5.04 (s, 2H), 6.91 (d, 2H), 7.11 (d, 2H), 7.40 (m, 5H); m/z^{M+1}: 258.2

### Step II

### Synthesis of N-{1-[4-(benzyloxy)benzyl]-2-hydroxyethyl}-2-chloroacetamide

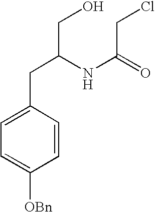

To a suspension of 2-amino-3-[4-(benzyloxy)phenyl]propan-1-ol hydrochloride (1.0 g, 3.40 mmol) in dichloromethane (30 ml) was added triethylamine (1.42 ml, 10.22 mmol) at 0-5° C. followed by chloro acetyl chloride (0.325 ml, 4.04 mmol). After completion of reaction, the reaction mixture was washed with 5% aq HCl solution, followed by brine solution, and then it was dried over sodium sulfate and concentrated to afford the title compound (0.820 g). ¹HNMR (CDCl₃ 400 MHz): δ 2.83 (m, 1H), 3.63 (dd, 1H), 3.69 (dd, 1H), 4.01 (d, 2H), 4.12 (m, 1H) 5.04 (s, 2H), 6.85 (d, 1), 6.93 (d, 2H), 7.15 (d, 2H), 7.40 (m, 5H); m/z^{M+1}: 334.2.

### Step III

### Preparation of 5-[4-(Benzyloxy)benzyl]morpholin-3-one

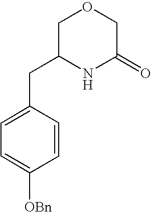

A solution of N-{1-[4-(benzyloxy)benzyl]-2-hydroxyethyl}-2-chloroacetamide (0.8 g, 2.40 mmol) in t-butanol (30 ml) was added over a period of 10 minutes to a suspension of potassium t-butoxide (0.4 g, 3.60 mmol) in t-butanol (20 ml) at 30° C., and was stirred for about four hours. After completion of the reaction, the reaction mixture was quenched with 5% aq. HCl solution and concentrated. The sticky mass thus obtained was neutralized with 5% NaHCO₃ solution and extracted with ethyl acetate. The organic layer thus obtained was dried over anhydrous sodium sulfate and concentrated to afford the title compound (0.7 g). ¹HNMR (CDCl₃ 400 MHz): δ 2.62 (m, 1H), 2.84 (dd, 1H), 3.55 (m, 1H), 3.62 (m, 1H), 3.92 (dd, 1H), 4.16 (d, 2H), 5.06 (s, 2H), 5.84 (bs, 1H), 6.94 (d, 2H), 7.11 (d, 2H), 7.42 (m, 5H); m/z^{M+1}: 293.3.

### Step IV

### Preparation of 5-(4-Hydroxybenzyl) morpholin-3-one

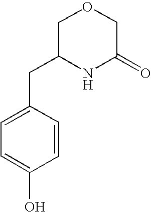

To a solution of 5-[4-(benzyloxy)benzyl]morpholin-3-one (0.7 g, 2.35 mmol) in methanol (100 ml) was added 10% Pd/C (0.100 g), and the reaction mixture was hydrogenated at a pressure of 40 psi, for 5 to 6 hours. The progress of the reaction was monitored by TLC, and upon completion, the solvent was evaporated under reduced pressure to afford a white solid (0.41 g). ¹HNMR (CDCl₃ 400 MHz): δ 2.63 (m, 1H), 2.81 (dd, 1H), 3.54 (m, 1H), 3.68 (m, 1H), 3.89 (dd, 1H), 4.16 (d, 2H), 5.86 (bs, 1H), 6.81 (d, 211), 7.05 (d, 2H); m/z^{M+1}: 209.3.

### Step V

### Preparation of 5-[4-(4-Nitrophenoxy)benzyl]morpholin-3-one

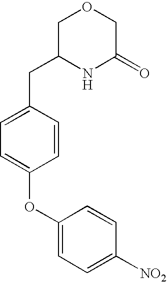

To a solution of 5-(4-hydroxybenzyl) morpholin-3-one (2.0 g, 9.66 mmol) and potassium carbonate (7.99 g, 57.9 mmol) in dimethylformamide (40 ml), was added 4-fluoronitrobenzene (1.63 g, 11.59 mmol). The reaction mixture was heated at 80° C. for 5 hours. Subsequently the reaction mixture was quenched with cold water (150 ml), then extracted with ethyl acetate, and the solvent was evaporated to give the desired product (3.1 g, 98.1%). ¹HNMR (CDCl₃, 400 MHz): δ 2.77 (q, 1H), 2.93 (s, 3H)), 3.15 (dd, 1H), 3.95 (m, 1H), 4.02 (m, 1H), 4.25 (t, 1H), 7.00 (dd, 2H), 7.07 (dd, 2H), 7.24 (m, 2H), 8.21 (d, 2H); m/z^{M+1}: 328.9

### Stage-VI

### Preparation of 4-[4-(4-Aminophenoxy)benzyl]-morpholin-3-one

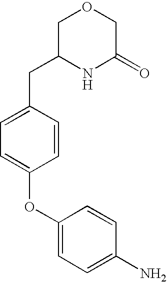

To a solution of 3-methyl-4-[4-(4-nitrophenoxy)benzyl]-1,3-oxazolidin-2-one (3.1 g, 9.45 mmol) in methanol (200 ml), was added 10% Pd/C (0.5 g), and the reaction mixture was hydrogenated at a pressure of 40 psi for 4 hours. After completion of reaction, the catalyst was filtered off, and the reaction mixture was concentrated to gave 4-[4-(4-aminophenoxy)benzyl]-morpholin-3-one (2.7 g, 96.4%). m/z^{M+1} 299.

### Stage-VII

### Preparation of 4-{4-[4-(Pyridin-2-ylamino)phenoxy]benzyl}-morpholin-3-one

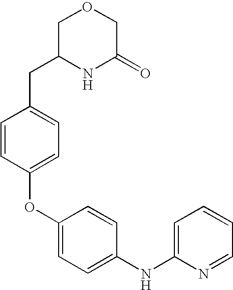

A solution of 4-[4-(4-aminophenoxy)benzyl]-morpholin-3-one (0.5 g, 1.67 mmol), and 2-chloro pyridine (0.95 ml, 10.06 mmol) was stirred at 130° C., under a nitrogen atmosphere for 20 hours. After completion of reaction, the reaction mixture was quenched with cold water, and was extracted with ethyl acetate and the solvent was evaporated to give the desired product. The crude product was purified by column chromatography (0.150 g, 23.8%). ¹HNMR (CDCl₃, 400 MHz): δ 2.67 (q, 1H), 2.86 (dd, 1H), 3.56 (q, 1H), 3.74 (m, 1H), 3.95 (dd, 1H), 4.19 (s, 2H), 5.86 (bs, 1H), 6.48 (s, 1H), 6.78 (m, 1H), 6.96 (dd, 1H) 7.00 (m, 4H), 7.12 (d, 2H), 7.32 (d, 2H), 7.49 (m, 1H), 8.18 (d, 1H); m/z^{M+1} 376.1

The Following Compounds were Prepared According to the Procedure Given in Example 1.

| Exp. | Structure | Analytical data |
|---|---|---|
| 2 | 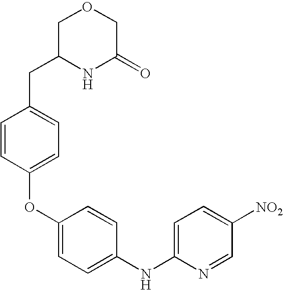 | Yield: 0.2 g; ¹HNMR. (CDCl₃, 400 MHz): δ 2.7 (q, 1 H), 2.88 (dd, 1 H), 3.6 (m, 1 H), 3.76 (m, 1 H), 3.93 (dd, 1 H), 4.19 (s, 2 H), 5.84 (bs, 1 H), 6.70 (d, 1 H), 7.00 (m, 5 H), 7.18 (d, 2 H), 7.36 (d, 2 H), 8.24 (d, 1 H), 9.08 (s, 1 H); m/z^{M+1} 421 |
| 3 | 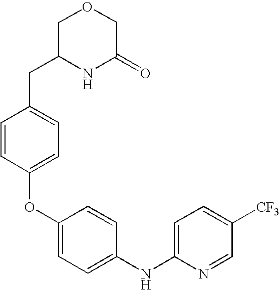 | Yield: 0.15 g; ¹HNMR(CDCl₃, 400 MHz): δ 2.69 (q, 1 H), 2.85 (dd, 1 H), 3.59 (q, 1 H), 3.74 (m, 1 H), 3.93 (dd, 1 H), 4.19 (s, 2 H), 5.94 (bs, 1 H) 6.75 (d, 1 H), 6.82 (d, 1 H), 7.00 (m, 4 H), 7.14 (d, 2 H), 7.35 (d, 2 H), 7.64 (d, 1 H), 8.41 (s, 1 H); m/z^{M+1}: 444.1 |
| 4 | 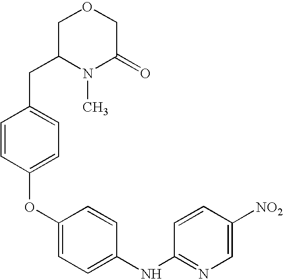 | Yield 0.120 g; ¹HNMR. (DMSO-d₆ , 400 MHz): δ 2.80 (q, 1 H), 2.91 (s, 3 H), 3.07 (dd, 1 H), 3.49 (m, 1 H), 3.63 (m, 2 H), 4.05 (s, 2 H), 6.87 (dd, 1 H) 6.96 (d, 2 H), 7.04 (d, 2 H), 7.26 (d, 2 H), 7.68 (d, 2 H), 8.29 (dd, 1 H), 9.01 (s, 1 H); m/z^{M+1}: 435.1 |
| 5 | 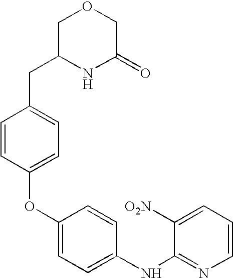 | Yield (0.300 g, ); ¹HNMR. (CDCl₃, 400 MHz): δ 2.72 (q, 1 H), 2.85 (dd, 1 H), 3.47 (m, 1 H), 3.66 (m, 2 H), 3.98 (s, 2 H), 7.0 (m, 4 H), 7.24 (d, 2 H), 7.61 (d, 2 H), 7.69 (m, 1 H), 8.12 (s, 1 H), 8.49 (m, 2 H), 9.94 (s, 1 H); m/z^{M+1}: 421.1 |
| 6 | 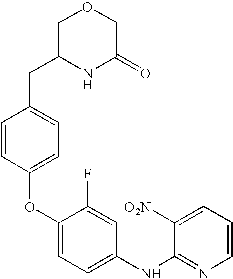 | Yield (0.300 g, ); ¹HNMR. (CDCl₃, 400 MHz): δ 2.68 (q, 1 H), 2.85 (dd, 1 H), 3.58 (m, 1 H), 3.7 (m, 1 H), 3.93 (dd, 1 H), 4.19 (s, 2 H), 5.87 (bs, 1 H), 6.9 (d, 1 H), 6.97 (d, 2 H), 7.13 (m, 3 H), 7.27 (m, 1 H), 7.85 (dd, 1 H), 8.5 (m, 2 H), 10.1 (s, 1 H); m/z^{M+1}: 439.1 |
| 7 | 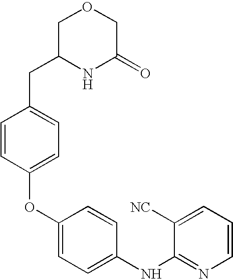 | Yield (0.135 g, ); ¹HNMR. (CDCl₃, 400 MHz): δ 2.70 (q, 1 H), 2.86 (dd, 1 H), 3.58 (q, 1 H), 3.75 (m, 1 H), 3.94 (dd, 2 H), 4.18 (s, 2 H), 6.0 (s, 1 H) 6.78 (d, 1 H), 7.0 (m, 5 H), 7.14 (d, 2 H), 7.55 (d, 2 H), 7.79 (d, 1 H), 8.36 (d, 1 H); m/z^{M+1}: 401.1 |
| 8 | 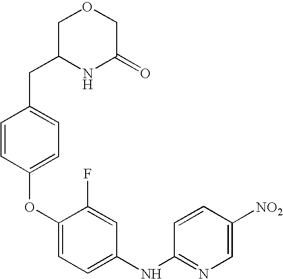 | Yield (0.050 g, ); ¹HNMR. (CDCl₃, 400 MHz): δ 2.68 (q, 1 H), 2.85 (dd, 1 H), 3.57 (m, 1 H), 3.73 (m, 1 H), 3.93 (dd, 1 H), 4.19 (s, 2 H), 5.9 (s, 1 H), 6.75 (d, 1 H), 6.97 (d, 2 H), 7.14 (m, 4 H), 7.20 (s, 1 H), 7.49 (dd, 1 H), 8.30 (dd, 1 H), 9.11 (d, 1 H); m/z^{M+1}: 439.1 |
| 9 | 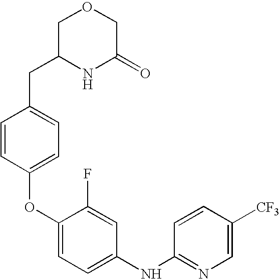 | Yield (0.100 g, ); ¹HNMR. (DMSO-d₆, 400 MHz): δ 2.71 (q, 1 H), 2.90 (dd, 1 H), 3.41 (m, 1 H), 3.61 (m, 2 H), 3.94 (s, 2 H), 6.88 (d, 2 H), 6.96 (d, 1 H), 7.19 (m, 3 H), 7.35 (d, 1 H), 7.9 (dd, 1 H), 8.0 (dd, 1 H), 8.11 (s, 1 H), 8.53 (s, 1 H), 9.84 (s, 1 H); m/z^{M+1}: 462.1 |
| 10 | 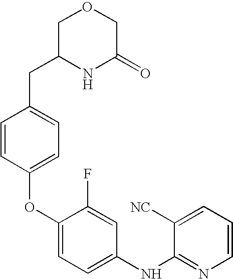 | Yield 0.150 g; ¹HNMR. (DMSO-d₆, 400 MHz): δ 2.70 (q, 1 H), 2.81 (dd, 1 H), 3.41 (m, 1 H), 3.62 (m, 2 H), 3.94 (s, 2 H), 6.89 (d, 2 H), 6.99 (m, 1 H), 7.14 (m, 1 H), 7.17 (m, 2 H), 7.45 (dd, 1 H), 7.73 (dd, 1 H) 8.13 (m, 2 H), 8.42 (d, 1 H), 9.32 (s, 1 H) m/z^{M+1}: 419.1 |
| 11 | 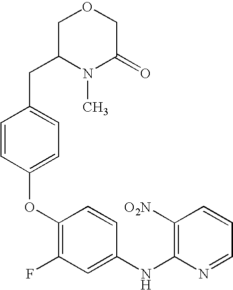 | Yield 0.600 g; ¹HNMR. (CDCl₃, 400 MHz): δ 2.95 (q, 1 H), 3.05 (s, 3 H), 3.08 (dd, 1 H), 3.29 (dd, 1 H) 3.65 (d, 1 H), 3.76 (d, 1 H), 4.23 (q, 2 H), 6.90 (m, 1 H) 6.94 (d, 2 H), 7.1 (m, 1 H), 7.16 (m, 3 H), 7.84 (dd, 1 H), 8.54 (m, 2 H), 10.17 (s, 1 H), m/z^{M+1}: 453.0 |
| 12 | 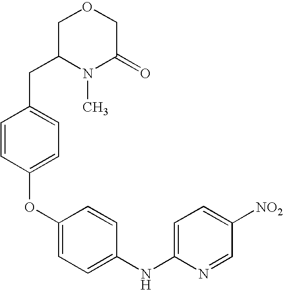 | Yield (0.300 g, ; ¹HNMR. (CDCl₃, 400 MHz): δ 2.95 (q, 1 H), 3.05 (s, 3 H), 3.08 (m, 1 H), 3.32 (dd, 1 H), 3.67 (d, 1 H), 3.76 (d, 1 H), 4.20 (q, 2 H), 6.69 (d, 1 H) 7.01 (m, 4 H), 7.19 (m, 3 H), 7.34 (d, 2 H), 8.24 (dd, 1 H), 9.08 (s, 1 H), m/z^{M+1}: 435.1 |
| 13 | 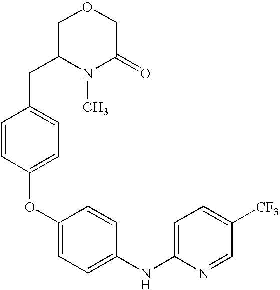 | Yield 0.80 g, ; ¹HNMR. (CDCl₃, 400 MHz): δ 2.94 (q, 1 H), 3.05 (s, 3 H), 3.10 (d, 1 H), 3.32 (d, 1 H), 3.66 (dd, 1 H), 3.76 (dd, 1 H), 4.25 (q, 2 H), 6.75 (d, 2 H) 7.01 (m, 4 H), 7.18 (d, 2 H), 7.33 (m, 2 H), 7.65 (dd, 1 H), 8.42 (s, 1 H), m/z^{M+1}: 458.1 |
| 14 | 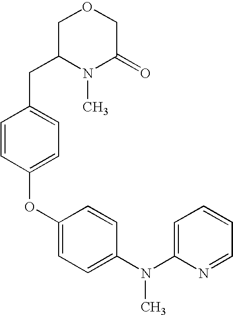 | Yield 0.150 g; ¹HNMR. (CDCl₃, 400 MHz): δ 2.95 (q, 1 H), 3.05 (s, 3 H), 3.08 (d, 1 H), 3.41 (d, 1 H), 3.49 (s, 3 H), 3.65 (dd, 1 H), 3.76 (d, 1 H), 4.25 (q, 2 H), 6.51 (d, 1 H) 6.70 (d, 1 H), 7.03 (m, 4 H), 7.21 (m, 4 H), 7.36 (m, 1 H), 8.24 (d, 1 H), m/z^{M+1}: 404.1 |
| 15 | 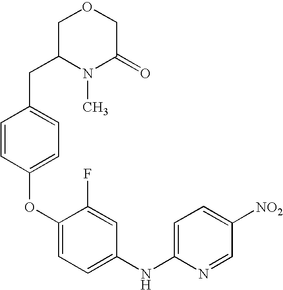 | Yield 0.92 g; ¹HNMR. (CDCl₃, 400 MHz): δ 2.94 (q, 1 H), 3.04 (s, 3 H), 3.06 (m, 1 H), 3.30 (d, 1 H), 3.66 (dd, 1 H), 3.76 (dd, 1 H), 4.20 (q, 2 H), 6.77 (d, 1 H) 6.95 (d, 2 H), 7.08 (m, 1 H), 7.17 (m, 3 H), 7.39 (s, 1 H), 7.52 (dd, 1 H), 8.28 (dd, 1 H), 9.12 (s, 1 H), m/z^{M+1}: 453.1 |
| 16 | 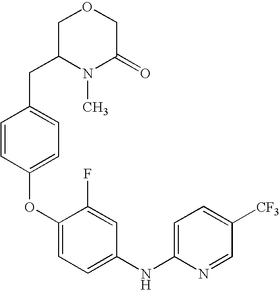 | Yield 0.900 g, ; ¹HNMR. (CDCl₃, 400 MHz): δ 2.93 (m, 1 H), 3.04 (s, 3 H), 3.08 (m, 1 H), 3.30 (d, 1 H), 3.62 (dd, 1 H), 3.65 (dd, 1 H), 4.20 (q, 2 H), 6.80 (d, 1 H) 6.93 (m, 3 H), 7.09 (m, 2 H), 7.15 (m, 2 H), 7.48 (dd, 1 H), 7.69 (dd, 1 H), 8.47 (s, 1 H), m/z^{M+1}: 476.1 |
| 17 | 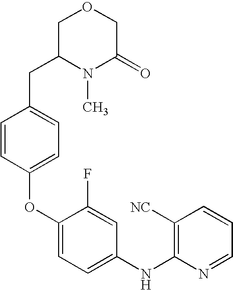 | Yield 0.700 g, ; ¹HNMR. (CDCl₃, 400 MHz): δ 2.91 (q, 1 H), 3.04 (s, 3 H), 3.08 (m, 1 H), 3.29 (d, 1 H), 3.61 (dd, 1 H), 3.76 (dd, 1 H), 4.25 (q, 2 H) 6.86 (m, 1 H), 6.94 (d, 2 H), 7.07 (m, 1 H), 7.13 (m, 4 H), 7.82 (m, 2 H), 8.41 (d, 1 H); m/z^{M+1}: 433.1 |
| 18 | 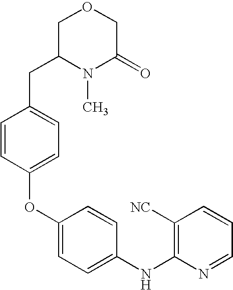 | Yield: 0.700 g, ; ¹HNMR. (CDCl₃, 400 MHz): δ 2.93 (q, 1 H), 3.04 (s, 3 H), 3.06 (d, 1 H), 3.30 (d, 1 H), 3.61 (dd, 1 H), 3.64 (dd, 1 H), 4.25 (q, 2 H), 6.79 (m, 1 H), 6.99 (m, 5 H), 7.16 (d, 2 H), 7.53 (d, 2 H), 7.79 (dd, 1 H), 8.36 (d, 1 H), m/z^{M+1}: 415.1 |
| 19 | 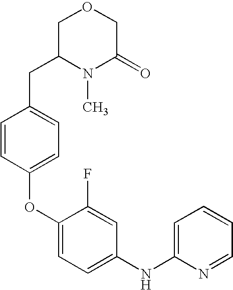 | Yield: 0.66 g, ; ¹HNMR. (CDCl₃, 400 MHz): δ 2.91 (q, 1 H), 3.04 (s, 3 H), 3.08 (d, 1 H), 3.28 (d, 1 H), 3.61 (dd, 1 H), 3.64 (dd, 1 H), 4.25 (q, 2 H), 6.56 (s, 1 H) 6.79 (m, 2 H), 6.92 (d, 2 H), 7.05 (d, 2 H), 7.13 (d, 2 H), 7.46 (m, 2 H), 8.24 (s, 1 H), m/z^{M+1}: 408.1 |
| 20 | 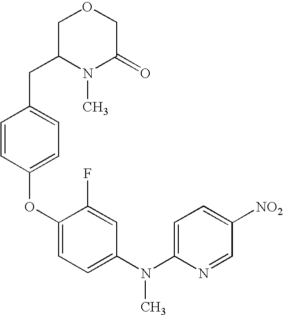 | Yield: 0.210 g; ¹HNMR. (CDCl₃, 400 MHz): δ 2.96 (q, 1 H), 3.04 (s, 3 H), 3.08 (m, 1 H), 3.32 (d, 1 H), 3.57 (s, 3 H), 3.66 (dd, 1 H), 3.77 (dd, 1 H), 4.20 (q, 2 H), 6.43 (dd, 1 H, ) 7.02 (m, 3 H), 7.12 (t, 2 H), 7.21 (d, 2 H), 8.11 (dd, 1 H), 9.12 (s, 1 H), m/z^{M+1}: 467.2 |
| 21 | 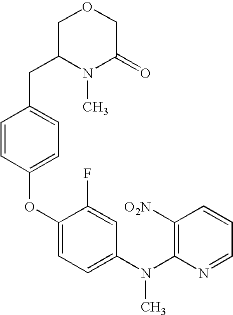 | Yield: 0.150 g; ¹HNMR. (CDCl₃, 400 MHz): δ 2.95 (m, 2 H), 3.04 (s, 3 H), 3.19 (d, 1 H), 3.59 (s, 3 H), 3.64 (d, 1 H), 3.74 (d, 1 H), 4.25 (q, 2 H), 6.78 (m, 1 H), 6.94 (m, 5 H), 7.16 (d, 2 H), 8.01 (dd, 1 H), 8.49 (d, 1 H), m/z^{M+1}: 467.2 |
| 22 | 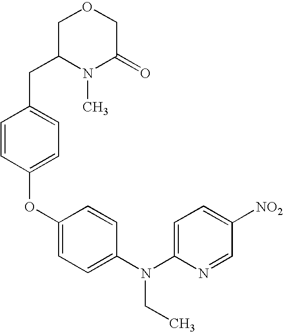 | Yield: 0.150 g; ¹HNMR. (CDCl₃, 400 MHz): δ 1.25 (t, 3 H), 2.98 (q, 1 H), 3.05 (s, 3 H), 3.10 (d, 1 H), 3.42 (d, 1 H), 3.67 (dd, 1 H), 3.76 (dd, 1 H), 4.06 (q, 2 H), 4.26 (q, 2 H), 6.19 (d, 1 H) 7.06 (m, 4 H), 7.17 (d, 2 H), 7.23 (m, 2 H), 8.01 (dd, 1 H), 9.10 (s, 1 H), m/z^{M+1}: 463.2 |
| 23 | 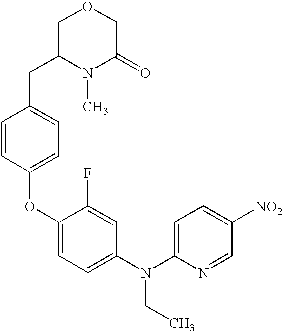 | Yield 0.190 g; ¹HNMR. (CDCl₃, 400 MHz): δ 1.27 (t, 3 H), 2.97 (q, 1 H), 3.04 (s, 3 H), 3.08 (d, 1 H), 3.45 (d, 1 H), 3.67 (d, 1 H), 3.76 (d, 1 H), 4.08 (q, 2 H), 4.25 (q, 2 H), 6.28 (d, 1 H) 7.00 (m, 3 H), 7.11 (m, 2 H), 7.22 (d, 2 H), 8.07 (dd, 1 H), 9.11 (s, 1 H), m/z^{M+1}: 481.3 |
| 24 | 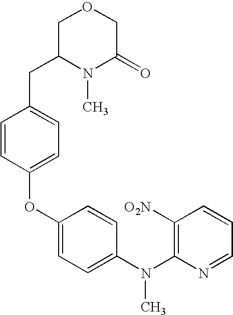 | Yield (0.203 g); ¹HNMR (CDCl₃, 400 MHz): δ 2.90 (q, 1 H), 2.93 (d, 1 H), 3.04 (s, 3 H), 3.31 (d, 1 H), 3.57 (s, 3 H), 3.65 (d, 1 H), 3.76 (d, 1 H), 4.2 (q, 2 H), 6.85 (m, 1 H), 6.94 (m, 4 H), 7.03 (m, 2 H), 7.17 (m, 2 H), 7.95 (dd, 1 H), 8.45 (d, 1 H) m/z^{M+1}: 448.9 |
| 25 | 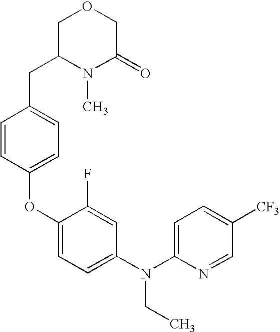 | Yield: 0.095 g, ; ¹HNMR. (CDCl₃, 400 MHz): δ 1.24 (t, 3 H), 2.96 (q, 1 H), 3.06 (s, 4 H), 3.11 (m, 1 H), 3.31 (m, 1 H), 3.64 (dd, 1 H), 3.77 (dd, 1 H), 4.01 (q, 2 H), 4.2 (q, 2 H), 6.38 (d, 1 H) 6.99 (m, 3 H), 7.09 (m, 2 H), 7.20 (m, 2 H), 7.48 (dd, 1 H), 8.44 (s, 1 H), m/z^{M+1}: 504 |
| 26 | 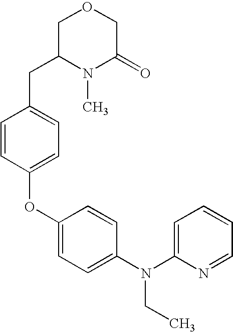 | Yield: 0.140 g; ¹HNMR. (CDCl₃, 400 MHz): δ 1.23 (t, 3 H), 2.95 (q, 1 H), 3.04 (s, 3 H), 3.1 (m, 1 H), 3.32 (m, 1 H), 3.64 (dd, 1 H), 3.78 (dd, 1 H), 3.98 (q, 2 H), 4.2 (q, 2 H), 6.32 (d, 1 H) 6.56 (t, 1 H), 7.03 (d, 4 H), 7.19 (d, 4 H), 7.28 (m, 1 H), 8.17 (d, 1 H); m/z^{M+1}: 418.0 |
| 27 | 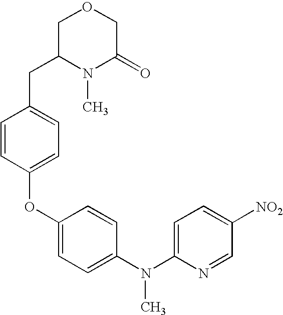 | Yield: 0.200 g; ¹HNMR. (CDCl₃, 400 MHz): δ 2.96 (m, 2 H), 3.05 (s, 3 H), 3.34 (d, 1 H), 3.56 (s, 3 H), 3.65 (dd, 1 H), 3.78 (dd, 1 H), 4.21 (q, 2 H), 6.35 (d, 1 H) 7.06 (m, 4 H), 7.23 (m, 4 H), 8.04 (dd, 1 H), 9.13 (s, 1 H), m/z^{M+1}: 448.8 |
| 28 | 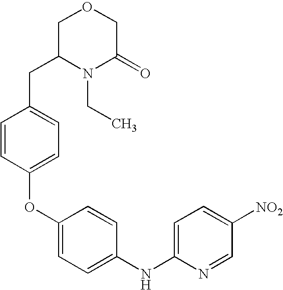 | Yield 0.637 g; ¹HNMR. (CDCl₃, 400 MHz): δ 1.24 (t, 3 H), 3.02 (m, 3 H), 3.37 (d, 1 H), 3.58 (dd, 1 H), 3.79 (dd, 1 H), 3.99 (m, 1 H), 4.20 (q, 2 H), 6.69 (d, 1 H) 7.02 (m, 4 H), 7.12 (bs, 1 H), 7.19 (d, 2 H), 7.36 (d, 2 H), 8.24 (dd, 1 H), 9.04 (s, 1 H), m/z^{M+1}: 448.9 |
| 29 | 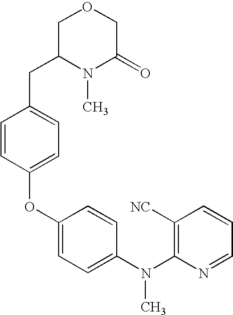 | Yield: 0.100 g; ¹HNMR. (CDCl₃, 400 MHz): δ 2.95 (q, 1 H), 3.04 (s, 3 H), 3.08 (m, 1 H), 3.31 (d, 1 H), 3.50 (s, 3 H), 3.64 (dd, 1 H), 3.76 (dd, 1 H), 4.20 (q, 2 H), 6.72 (m, 1 H), 7.04 (m, 4 H), 7.17 (d, 2 H), 7.25 (m, 2 H), 7.66 (d, 1 H), 8.40 (d, 1 H); m/z^{M+1}: 429.0 |
| 30 | 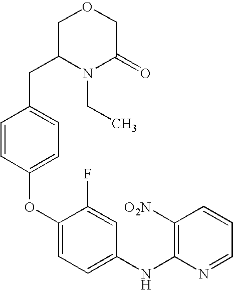 | Yield: 0.810 g; ¹HNMR. (CDCl₃, 400 MHz): δ 1.22 (t, 3 H), 3.00 (m, 3 H), 3.34 (d, 1 H), 3.56 (dd, 1 H), 3.78 (dd, 1 H), 3.98 (m, 1 H), 4.20 (q, 2 H), 6.91 (m, 3 H), 7.08 (m, 1 H), 7.16 (m, 2 H), 7.25 (s, 1 H), 7.85 (dd, 1 H), 8.53 (m, 2 H), 10.17 (s, 1 H): m/z^{M+1}: 466.9. |
| 31 | 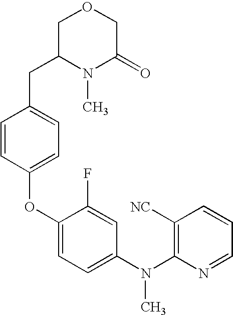 | Yield: 0.21 g; ¹HNMR. (CDCl₃, 400 MHz): δ 2.91 (q, 1 H), 3.03 (s, 3 H), 3.07 (m, 1 H), 3.29 (d, 1 H), 3.51 (s, 3 H), 3.60 (dd, 1 H), 3.75 (dd, 1 H), 4.19 (q, 2 H), 6.79 (m, 1 H) 7.03 (m, 3 H), 7.1 (m 4 H), 7.71 (d, 1 H), 8.43 (s, 1 H), m/z^{M+1}: 446.9. |
| 32 | 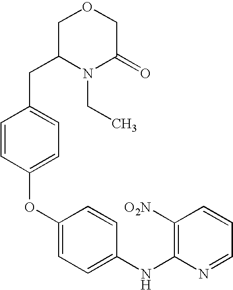 | Yield: 0.05 g; ¹HNMR. (CDCl₃ , 400 Mhz): δ 1.23 (t, 3 H), 3.00 (m, 3 H), 3.34 (m, 1 H), 3.56 (d, 1 H), 3.8 (d, 1 H), 3.99 (m, 1 H), 4.20 (q, 2 H), 6.84 (dd, 1 H), 7.02 (m, 4 H), 7.18 (d, 2 H), 7.59 (d, 2 H), 8.48 (d, 1 H), 8.52 (d, 1 H), 10.07 (s, 1 H): m/z^{M+1}: 449 |
| 33 | 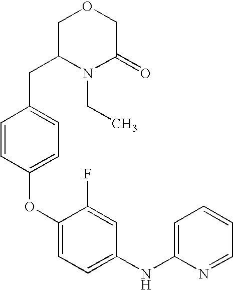 | Yield: 0.90 g; ¹HNMR. (CDCl₃, 400 MHz): δ 1.22 (t, 3 H), 2.99 (m, 3 H), 3.34 (d, 1 H), 3.54 (dd, 1 H), 3.78 (dd, 1 H), 3.98 (m, 1 H), 4.19 (q, 2 H), 6.8 (m, 3 H) 6.94 (d, 2 H), 7.08 (m, 2 H), 7.15 (d, 2 H), 7.48 (dd, 1 H), 7.71 (dd, 1 H), 8.48 (s, 1 H): m/z^{M+1}: 422.2. |
| 34 | 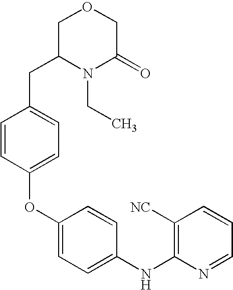 | Yield: 0.750 g; 1HNMR. (CDCl₃, 400 MHz): δ 1.22 (t, 3 H), 2.99 (m, 3 H), 3.34 (d, 1 H), 3.58 (dd, 1 H), 3.78 (dd, 1 H), 3.99 (m, 1 H), 4.19 (q, 2 H), 6.79 (d, 1 H) 7.00 (m, 5 H), 7.16 (d, 2 H), 7.53 (d, 2 H), 7.78 (d, 1 H), 8.36 (d, 1 H): m/z^{M+1}: 429.0. |
| 35 | 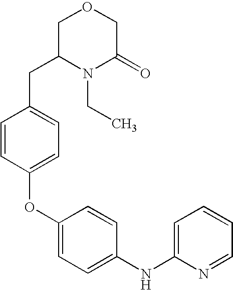 | Yield: 0.90 g; ¹HNMR. (CDCl₃, 400 MHz): δ 1.22 (t, 3 H), 3.00 (m, 3 H), 3.34 (d, 1 H), 3.53 (dd, 1 H), 3.78 (dd, 1 H), 3.99 (m, 1 H), 4.19 (q, 2 H), 6.48 (s, 1 H), 6.75 (m, 2 H), 6.98 (m, 4 H), 7.15 (d, 2 H), 7.33 (d, 2 H), 7.48 (m, 1 H), 8.19 (s, 1 H): m/z^{M+1}: 404.4. |
| 36 | 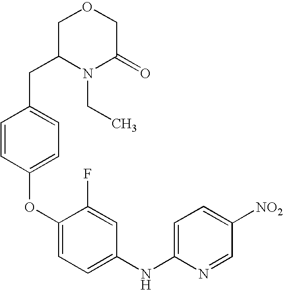 | Yield: 0.830 g; ¹HNMR. (DMSO-d₆, 400 MHz): δ 1.09 (t, 3 H), 2.80 (q, 1 H), 2.99 (m, 2 H), 3.53 (m, 3 H), 3.72 (m, 1 H), 4.01 (q, 2 H), 6.92 (m, 3 H), 7.23 (m, 3 H), 7.41 (d, 1 H), 7.98 (d, 1 H), 8.33 (d, 1 H), 9.08 (s, 1 H), 10.32 (s, 1 H): m/z^{M+1}: 466.9. |
| 37 | 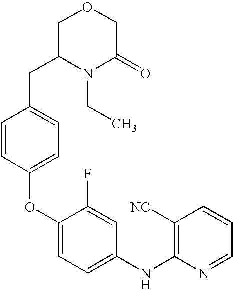 | Yield: 0.812 g; ¹HNMR. (DMSO-d₆, 400 MHz): δ 1.09 (t, 3 H), 2.79 (t, 1 H), 3.01 (d, 2 H), 3.52 (m, 3 H), 3.73 (m, 1 H), 4.03 (q, 2 H), 6.91 (d, 2 H), 6.99 (m, 1 H), 7.20 (t, 1 H), 7.25 (d, 2 H), 7.43 (d, 1 H), 7.72 (dd, 1 H), 8.14 (dd, 1 H), 8.42 (d, 1 H), 9.34 (s, 1 H); m/z^{M+1}: 446.9. |
| 38 | 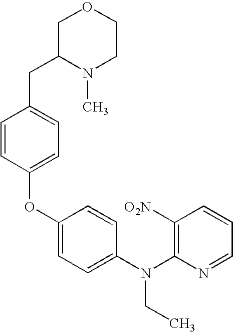 | Yield: 0.150 g; ¹HNMR. (DMSO-d₆, 400 MHz): δ 1.15 (t, 3 H), 2.20 (m, 2 H), 2.31 (s, 3 H), 2.38 (m, 1 H), 2.65 (dd, 1 H), 2.94 (dd, 1 H), 3.12 (t, 1 H), 3.31 (m, 1 H), 3.4 (m, 1 H), 3.63 (dd, 1 H), 4.07 (q, 2 H), 6.91 (m, 4 H), 6.99 (m, 1 H), 7.07 (d, 2 H), 7.20 (d, 2 H), 8.08 (dd, 1 H), 8.51 (d, 1 H); m/z^{M+1}: 449.1 |
| 39 | 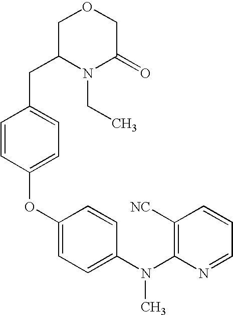 | Yield: 0.22 g; ¹HNMR. (CDCl3, 400 MHz): δ 1.22 (t, 3 H), 2.88 (s, 1 H), 2.96 (s, 1 H), 3.00 (m, 1 H), 3.34 (d, 1 H), 3.49 (s, 3 H), 3.55 (d, 1 H), 3.78 (d, 1 H), 3.98 (m, 1 H), 4.19 (q, 2 H), 6.72 (m, 1 H), 7.05 (m, 4 H), 7.17 (d, 2 H), 7.25 (m, 2 H), 7.66 (dd, 1 H), 8.40 (d, 1 H); m/z^{M+1}: 443.2 |
| 40 | 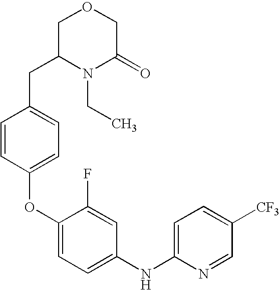 | Yield 0.81 g; ¹HNMR. (CDCl₃, 400 MHz): δ 1.22 (t, 3 H), 3.00 (m, 3 H), 3.34 (d, 1 H), 3.54 (dd, 1 H), 3.77 (dd, 1 H), 3.98 (m, 1 H), 4.19 (q, 2 H), 6.75 (s, 1 H), 6.80 (d, 1 H), 6.94 (d, 2 H), 7.08 (m, 2 H), 7.15 (d, 2 H), 7.48 (dd, 1 H), 7.71 (dd, 1 H), 8.48 (s, 1 H); m/z^{M+1}: 489.9 |
| 41 | 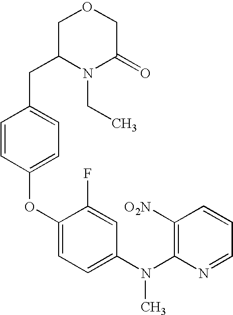 | Yield 0.19 g; ¹HNMR. (CDCl3, 400 MHz): δ 1.23 (t, 3 H), 2.98 (m, 4 H), 3.34 (d, 1 H), 3.57 (s, 3 H), 3.78 (d, 1 H), 3.99 (m, 1 H), 4.23 (q, 2 H), 6.78 (d, 1 H), 6.95 (m, 5 H), 7.15 (d, 2 H), 8.0 (dd, 1 H), 8.48 (d, 1 H); m/z^{M+1}: 481.1 |
| 42 | 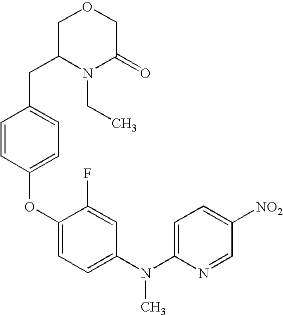 | Yield 0.210 g; ¹HNMR. (CDCl3, 400 MHz): δ 1.23 (t, 3 H), 2.99 (m, 4 H), 3.34 (d, 1 H), 3.58 (s, 3 H), 3.78 (d, 1 H), 4.01 (m, 1 H), 4.23 (q, 2 H), 6.43 (d, 1 H), 7.01 (d, 3 H), 7.12 (m, 2 H), 7.21 (d, 2 H), 8.10 (dd, 1 H), 9.12 (s, 1 H); m/z^{M+1}: 481.2. |
| 43 | 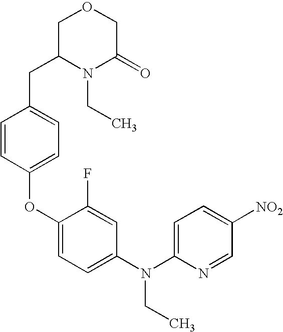 | Yield 0.250 g; ¹HNMR. (DMSO-d₆, 400 MHz): δ 1.09 (t, 3 H), 1.17 (t, 3 H), 2.83 (t, 1 H), 3.01 (m, 2 H), 3.56 (m, 3 H), 3.73 (m, 1 H), 4.03 (m, 4 H), 6.46 (dd, 1 H), 7.06 (d, 2 H), 7.22 (m, 2 H), 7.31 (m, 2 H), 7.56 (dd, 1 H), 8.16 (dd, 1 H), 9.0 (s, 1 H); m/z^{M+1}: 495.2. |
| 44 | 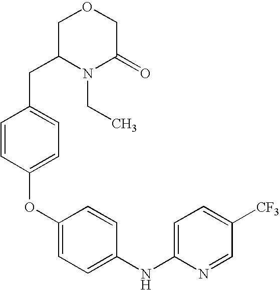 | Yield 0.800 g; ¹HNMR. (CDCl3, 400 MHz): δ 1.22 (t, 3 H), 2.98 (m, 1 H), 3.03 (q, 2 H), 3.36 (d, 1 H), 3.57 (dd, 1 H), 3.79 (dd, 1 H), 3.98 (m, 1 H), 4.23 (q, 2 H), 6.76 (d, 1 H), 6.98 (m, 4 H), 7.17 (d, 3 H), 7.35 (d, 2 H), 7.64 (d, 1 H), 8.41 (s, 1 H); m/z^{M+1}: 472.1 |
| 45 | 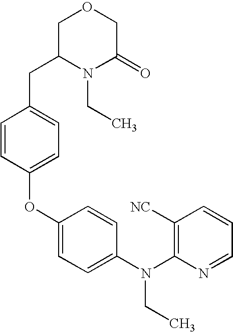 | Yield: 0.240 g; ¹HNMR. (CDCl3, 400 MHz): δ 1.25 (t, 6 H), 2.98 (m, 1 H), 3.00 (q, 2 H), 3.34 (d, 1 H), 3.57 (dd, 1 H), 3.78 (dd, 1 H), 4.01 (m, 3 H), 4.23 (q, 2 H), 6.67 (d, 1 H), 7.06 (m, 4 H), 7.17 (d, 2 H), 7.24 (d, 2 H), 7.63 (dd, 1 H), 8.37 (d, 1 H), : m/z^{M+1}: 457.1 |
| 46 | 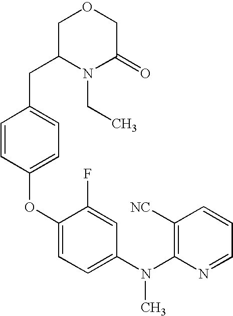 | Yield: 0.200 g; ¹HNMR. (CDCl3, 400 MHz): δ 1.21 (t, 3 H), 2.99 (m, 3 H), 3.25 (d, 1 H), 3.51 (s, 3 H), 3.56 (m, 1 H), 3.77 (d, 1 H), 4.01 (m, 1 H), 4.19 (q, 2 H), 6.80 (m, 1 H), 7.04 (m, 3 H), 7.10 (m, 2 H), 7.15 (m, 2 H), 7.71 (d, 1 H), 8.44 (d, 1 H); m/z^{M+1}: 461.1. |
| 47 | 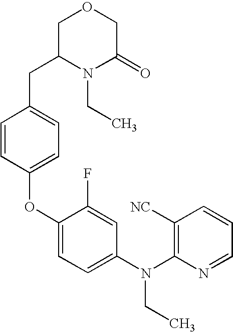 | Yield: 0.160 g; ¹HNMR. (CDCl3, 400 MHz): δ 1.25 (m, 6 H), 2.98 (m, 3 H), 3.34 (d, 1 H), 3.51 (dd, 1 H), 3.75 (dd, 1 H), 4.04 (m, 3 H), 4.23 (q, 2 H), 6.75 (d, 1 H), 7.02 (m, 3 H), 7.13 (m, 4 H), 7.66 (d, 1 H), 8.4 (d, 1 H); m/z^{M+1}: 475.2 |
| 48 | 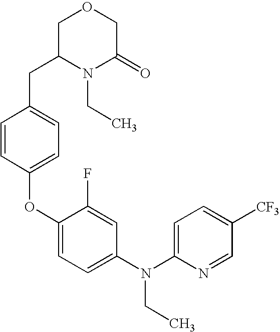 | Yield: 0.240 g; 1HNMR. (DMSO-d₆, 400 MHz): δ 1.09 (t, 3 H), 1.15 (t, 3 H), 2.82 (q, 1 H), 3.01 (m, 2 H), 3.56 (m, 3 H), 3.72 (m, 1 H), 4.01 (m, 4 H), 6.52 (d, 1 H), 7.03 (d, 2 H), 7.18 (m, 2 H), 7.3 (d, 2 H), 7.49 (dd, 1 H), 7.70 (dd, 1 H), 8.48 (s, 1 H), m/z^{M+1}: 518.1 |
| 49 | 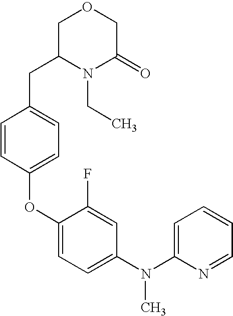 | Yield: 0.120 g; ¹HNMR. (DMSO-d₆, 400 MHz): δ 1.09 (t, 3 H), 2.75 (q, 1 H), 3.01 (q, 2 H), 3.38 (s, 3 H), 3.54 (m, 3 H), 3.75 (m, 1 H), 4.01 (q, 2 H), 6.72 (m, 2 H), 6.98 (d, 2 H), 7.15 (m, 2 H), 7.28 (d, 2 H), 7.39 (dd, 1 H), 7.50 (d, 1 H), 8.18 (d, 1 H); m/z^{M+1}: 436.1 |
| 50 | 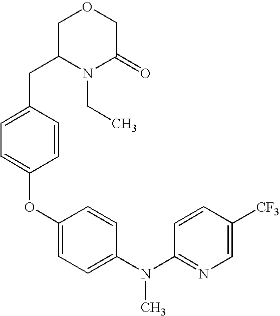 | Yield 0.130 g; ¹HNMR. (CDCl3, 400 MHz): δ 1.23 (t, 3 H), 2.99 (m, 3 H), 3.37 (d, 1 H), 3.48 (s, 3 H), 3.57 (d, 1 H), 3.79 (d, 1 H), 4.00 (m, 1 H), 4.19 (q, 2 H), 6.43 (dd, 1 H), 7.04 (m, 4 H), 7.2 (m, 4 H), 7.46 (dd, 1 H), 8.44 (s, 1 H); m/z^{M+1}: 486.1 |
| 51 | 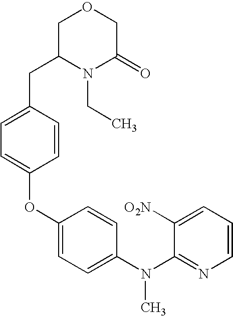 | Yield: 0.120 g; ¹HNMR. (CDCl3, 400 MHz): δ 1.22 (t, 3 H), 2.99 (m, 1 H), 3.02 (m, 3 H), 3.35 (d, 1 H), 3.55 (s, 3 H), 3.78 (dd, 1 H), 3.97 (m, 1 H), 4.20 (q, 2 H), 6.85 (m, 1 H), 6.94 (m, 4 H) 7.04 (d, 2 H), 7.17 (d, 2 H), 7.95 (d, 1 H), 8.44 (s, 1 H); m/z^{M+1}: 463.1 |
| 52 | 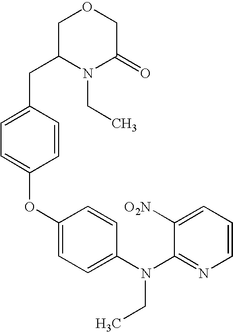 | Yield: 0.100 g; ¹HNMR. (CDCl3, 400 MHz): δ 1.22 (t, 3 H), 1.25 (t, 3 H), 3.02 (m, 3 H), 3.34 (d, 1 H), 3.57 (dd, 1 H), 3.78 (d, 1 H), 4.00 (m, 1 H), 4.14 (m, 3 H), 4.26 (d, 1 H), 6.80 (m, 1 H), 6.94 (m, 4 H), 7.01 (d, 2 H), 7.17 (d, 2 H), 7.90 (d, 1 H), 8.43 (s, 1 H); m/z^{M+1}: 477.2. |
| 53 | 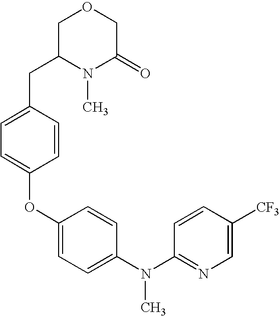 | Yield: 0.210 g; ¹HNMR. (DMSO-d₆, 400 MHz): δ 2.73 (q, 1 H), 2.91 (s, 3 H), 3.31 (dd, 1 H), 3.42 (s, 3 H), 3.5 (m, 1 H), 3.63 (q, 2 H), 4.04 (q, 2 H), 6.50 (dd, 1 H) 7.06 (m, 4 H), 7.33 (m, 4 H), 7.70 (d, 1 H), 8.48 (s, 1 H); m/z^{M+1}: 472.1. |
| 54 | 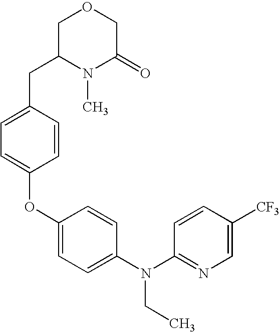 | Yield: 0.110 g; ¹HNMR. (DMSO-d₆, 400 MHz): δ 1.13 (t, 3 H) 2.87 (q, 1 H), 2.91 (s, 3 H), 3.25 (dd, 1 H), 3.52 (d, 1 H), 3.61 (m, 2 H), 3.95 (q, 2 H), 4.04 (s, 2 H), 6.49 (dd, 1 H) 7.08 (m, 4 H), 7.32 (d, 4 H), 7.82 (dd, 1 H), 8.5 (s, 1 H); m/z^{M+1}: 486.1. |

### EXAMPLE 55

### Synthesis of 5-Nitro N-{4-[4-{(4-methylmorpholin-3-yl)methyl}phenoxy]phenyl}-pyridin-2-amine

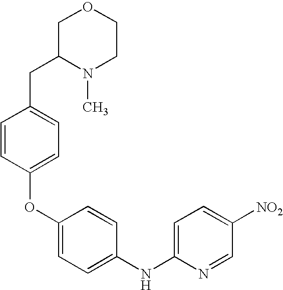

### Step I: Synthesis of 5-[4-(Benzyloxy)benzyl]-4-methylmorpholin-3-one

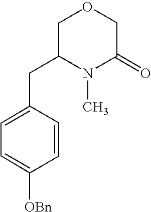

To the suspension of sodium hydride (0.8 g, 20.2 mmol) (60% in mineral oil) in tetrahydrofuran (20 ml) was added a solution of 5-[4-(benzyloxy)benzyl]morpholin-3-one (4.0 g, 13.4 mmol) in 20 ml tetrahydrofuran at 0° C. and stirred for 30 minutes. To this added methyl iodide (10.2 ml, 73.4 mmol) and stirred for 2 hours at same temperature. The reaction mixture was quenched with saturated aqueous ammonium chloride solution and extracted with ethyl acetate. The organic layer was dried over anhydrous sodium sulphate and concentrated to yield the title compound. Yield 3.8 g, m/z^{M+1}: 311

### Step II: Synthesis of 3-[4-(Benzyloxy)benzyl]-4-methylmorpholine

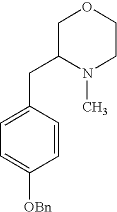

To the suspension of lithium aluminium hydride (1.83 g, 48.2 mmol) in tetrahydrofuran (20 ml) was added a solution of 5-[4-(benzyloxy)benzyl]-4-methylmorpholin-3-one (5 g, 16 mmol) in tetrahydrofuran (25 ml) at 0° C. and stirred the reaction mixture for 2 hour. The reaction mixture was quenched with saturated aqueous ammonium chloride solution and extracted with ethyl acetate. The organic layer was dried over anhydrous sodium sulphate and concentrated to yield the title compound. Yield 1.5 g, m/z^{M+1}: 298

### Step III: Synthesis of 4-[(4-Methylmorpholin-3-yl)methyl]phenol (Prepared According to Procedure Described in Example 1 (Step IV)

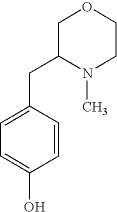

### Step IV: Synthesis of 4-Methyl-3-[4-(4-nitrophenoxy)benzyl]morpholine (Prepared According to Procedure Described in Example 1 (Step V)

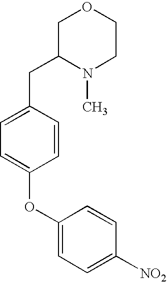

### StepV: Synthesis of 4-{4-[(4-Methylmorpholin-3-yl)methyl]phenoxy}aniline (Prepared According to Procedure Described in Example 1 (Step VI)

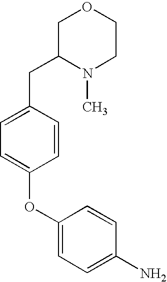

### StepVI: Synthesis of 5-Nitro N-{4-[4-{(4-methylmorpholin-3-yl)methyl}phenoxy]phenyl}-pyridin-2-amine (Prepared According to Procedure Described in Example 1 (Step VI)

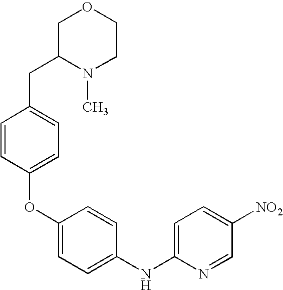

Yield: 0.800 g; ¹HNMR. (DMSO-d₆, 400 MEN): δ 2.22 (m, 2H), 2.31 (s, 3H), 2.37 (m, 1H), 2.65 (dd, 1H), 3.00 (dd, 1H), 3.11 (m, 1H), 3.38 (m, 1H), 3.47 (m, 1H), 3.62 (d, 1H), 6.90 (m, 3H) 7.02 (d, 2H), 7.19 (d, 2H), 7.69 (d, 2H), 8.28 (d, 1H), 9.01 (s, 1H); m/z^{M+1}: 421.2

The Following Compounds were Prepared According to the Procedure Given in Example 55.

| Exp. | Structure | Analytical data |
|---|---|---|
| 56 | 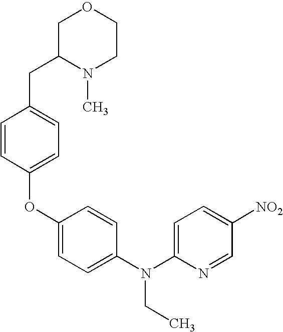 | Yield (0.250 g; ¹HNMR. (DMSO-d₆, 400 MHz): δ 1.16 (t, 3 H), 2.24 (m, 2 H), 2.32 (s, 3 H), 2.40 (m, 1 H), 2.67 (d, 1 H), 2.99 (dd, 1 H), 3.15 (t, 1 H), 3.40 (dd, 1 H), 3.49 (dd, 1 H), 3.65 (dd, 1 H), 4.01 (q, 2 H), 6.32 (d, 1 H) 7.04 (d, 2 H), 7.10 (d, 2 H), 7.26 (d, 2 H), 7.33 (d, 2 H), 8.12 (dd, 1 H), 9.02 (s, 1 H); m/z^{M+1}: 448.9 |
| 57 | 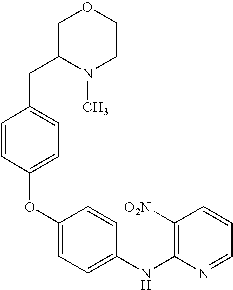 | Yield (1.2 g; ¹HNMR. (DMSO-d₆, 400 MHz): δ 2.23 (m, 2 H), 2.31 (s, 3 H), 2.36 (m, 1 H), 2.66 (d, 1 H), 2.97 (dd, 1 H), 3.13 (m, 1 H), 3.38 (dd, 1 H), 3.48 (m, 1 H), 3.62 (dd, 1 H), 6.98 (m, 5 H), 7.22 (m, 2 H), 7.61 (m, 2 H), 8.48 (d, 1 H), 8.53 (m, 1 H), 9.94 (s, 1 H), m/z^{M+1}: 421.2 |

### Protocols for Biological Testing

### Glucose Uptake Assay Using 3T3-L1 Cells

3T3-L1 cells were differentiated by the addition of differentiation cocktail (72 μg/ml insulin, 0.5 mM IBMX, 400 ng/ml Dexamethasone) for 4 days and later fed with media without differentation cocktail for 7-8 days. After differentiation the cells were incubated with either the reference compound BLX-1002 or compounds listed in the table 1 at 1 μM concentrations for 72 hours and carried out the glucose uptake assay for 10 min by the addition of KRP buffer supplemented with 2.5 μCi/ml ¹⁴C deoxy glucose. Stimulation Index is defined as the amount of ¹⁴C Deoxyglucose uptake induced by 1 μM of BLX-1002 incubated for 72 hours in an assay condition as per protocol described above with differentiated 3T3-L1 adipocytes. Values of compounds mentioned in table-1 are with reference to stimulation index of reference compound BLX-1002.

TABLE 1
Effect of compounds on glucose uptake assay in 3T3-L1 cells

| Exp. No. | Stimulation Index |
|---|---|
| | 1.0 |
| 1 | 0.88 |
| 2 | 1.06 |
| 6 | 1.06 |
| 7 | 1.05 |
| 8 | 0.66 |
| 9 | 0.92 |
| 10 | 0.82 |
| 11 | 1.14 |
| 12 | 1.18 |
| 13 | 1.00 |

1. Novel heterocyclic analogs of biphenyl ethers of the general formula (I) and their pharmaceutically acceptable salts and compositions, 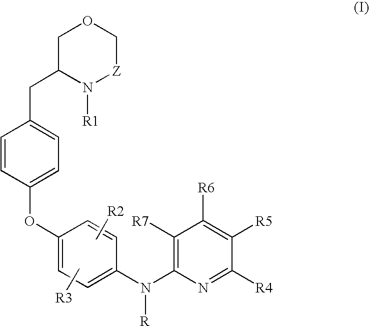
wherein Z represents CH₂, C═O; suitable groups represented by R and R₁ are same or different and independently represent hydrogen, alkyl, alkenyl, substituted or unsubstituted groups selected from (C₁-C₄) alkyl groups comprising methyl, ethyl, propyl, isopropyl, n-butyl, isobutyl, t-butyl and the like; substituted or unsubstituted linear or branched (C₂-C₇) alkenyl groups comprising ethenyl, propenyl, butenyl and the like; aryl groups comprising phenyl, naphthyl and the like, the aryl group are optionally substituted; aryloxy, substituted or unsubstituted linear or branched (C₂-C₅) alkoxy groups comprising methoxy, ethoxy, propoxy, n-butoxy, isobutoxy, t-butoxy and the like; COR₈, where R₈ represents substituted or unsubstituted groups selected from (C₁-C₄) alkyl groups comprising methyl, ethyl, propyl, isopropyl, n-butyl, isobutyl, t-butyl and the like; substituted or unsubstituted linear or branched (C₂-C₅) alkenyl groups comprising ethenyl, propenyl, butenyl and the like; aryl groups comprising phenyl, naphthyl and the like, the aryl group is optionally substituted; aryloxy, substituted or unsubstituted linear or branched (C₂-C₅) alkoxy groups comprising methoxy, ethoxy, propoxy, n-butoxy, isobutoxy, t-butoxy and the like.
Suitable groups represented by R₂ and R₃ are selected from hydrogen, halogen atoms comprising fluorine, chlorine, bromine or iodine; hydroxy, nitro, cyano, formyl, amino, substituted or unsubstituted linear or branched (C₁-C₄) alkyl groups comprising methyl, ethyl, n-propyl, isopropyl, n-butyl, isobutyl, t-butyl and the like; haloalkyl groups comprising chloromethyl, chloroethyl, trifluoromethyl, trifluoroethyl, dichloromethyl, dichloroethyl, trichloromethyl, difluoromethyl, and the like, which are optionally substituted; alkoxy groups comprising methoxy, ethoxy, n-propoxy, isopropoxy and the like, which are optionally substituted.
R₄, R₅, R₆ and R₇ are same or different and independently represent hydrogen, halogen atoms comprising fluorine, chlorine, bromine or iodine; hydroxy, nitro, cyano, formyl, amino, azido, hydrazine; substituted or unsubstituted groups selected from linear or branched (C₁-C₄) alkyl groups comprising methyl, ethyl, n-propyl, isopropyl, n-butyl, isobutyl, t-butyl and the like; haloalkyl groups comprising chloromethyl, chloroethyl, trifluoromethyl, trifluoroethyl, dichloromethyl, dichloroethyl, trichloromethyl, difluoromethyl, and the like, which are optionally substituted; alkoxy groups comprising methoxy, ethoxy, n-propoxy, isopropoxy and the like, which may be substituted; monoalkylamino groups comprising —NHCH₃, —NHC₂H₅, —NHC₃H₇, —NHC₆H₁₃, and the like, which may be substituted; dialkylamino groups comprising —N(CH₃)₂, —NCH₃(C₂H₅), —N(C₂H₅)₂ and the like, which are optionally substituted; carboxylic acids or its derivatives comprising esters or amides; acylamino groups comprising —NHC(═O)CH₃, —NHC(═O)C₂H₅, —NHC(═O)C₃H₇, —NHC(═O)C₆H₁₃, and the like, which are optionally substituted; alkylsulfonyl groups comprising methylsulfonyl, ethylsulfonyl, n-propylsulfonyl, iso-propylsulfonyl and the like, the alkylsulfonyl group is optionally substituted; arylsulfonyl groups comprising phenylsulfonyl or naphthylsulfonyl, the arylsulfonyl group is optionally substituted; alkylsulfinyl groups comprising methylsulfinyl, ethylsulfinyl, n-propylsulfinyl, iso-propylsulfinyl and the like, the alkylsulfinyl group is optionally substituted; arylsulfinyl groups comprising phenylsulfinyl or naphthylsulfinyl, the arylsulfinyl group is optionally substituted; alkylthio groups comprising methylthio, ethylthio, n-propylthio, iso-propylthio and the like, the alkylthio group is optionally substituted; alkoxycarbonyl groups comprising methoxycarbonyl, ethoxycarbonyl, n-propoxycarbonyl, isopropoxycarbonyl and the like, the alkoxycarbonyl group is optionally substituted; aryloxycarbonyl groups comprising phenoxycarbonyl, napthoxycarbonyl, and the like, the aryloxycarbonyl group is optionally substituted; alkoxyalkyl groups comprising methoxymethyl, ethoxymethyl, methoxyethyl, ethoxyethyl and the like, which are optionally substituted; sulfamoyl; carboxylic acid and its derivatives. Suitable substituents on the groups represented by R₁, R₂, R₃ R₄, R₅, R₆, R₇, and R₈ are selected from nitro, cyano, hydroxy, halo, formyl, azido, alkyl, alkoxy, acyl, haloalkyl, amino, hydrazine, monoalkylamino, dialkylamino, acylamino, alkylsulfonyl, alkylsulfinyl, arylsulfonyl, arylsulfinyl, alkylthio, arylthio, alkoxycarbonyl, aryloxycarbonyl, alkoxyalkyl, sulfamoyl, carboxylic acid and its derivatives.

2. Novel heterocyclic analogs of biphenyl ethers as claimed in claim 1, are selected from a group comprising of:
1). 5-{4-[4-(Pyridin-2-ylamino)phenoxy]benzyl}morpholin-3-one;
2). 5-(4-{4-[(5-Nitropyridin-2-yl)amino]phenoxy}benzyl)morpholin-3-one;
3). 5-(4-{4-[5-(Trifluoromethyl)pyridin-2-yl)amino]phenoxy}benzyl)morpholin-3-one;
4). 4-Methyl-5-(4-{4-[(5-nitropyridin-2-yl)amino]phenoxy}benzyl)morpholin-3-one;
5). 5-(4-{4-[(3-Nitropyridin-2-yl)amino]phenoxy}benzyl)morpholin-3-one;
6). 5-{4-[2-Fluoro-4-(3-nitropyridin-2-ylamino)phenoxy]benzyl}morpholin-3-one;
7). 2-[(4-{4-[(5-Oxomorpholin-3-yl)methyl]phenoxy}phenyl)amino]nicotinonitrile;
8). 5-{4-[2-Fluoro-4-(5-nitropyridin-2-ylamino)phenoxy]benzyl}morpholin-3-one;
9). 5-{4-[2-Fluoro-4-(5-(trifluoromethyl)pyridin-2-ylamino)phenoxy]benzyl}morpholin-3-one;
10). 2-[(3-Fluoro-4-{4-[(5-oxomorpholin-3-yl)methyl]phenoxy}phenyl)amino]nicotinonitrile;
11). 4-Methyl-5-{4-[2-fluoro-4-(3-nitropyridin-2-ylamino)phenoxy]benzyl}morpholin-3-one;
12). 4-Methyl-5-{4-[4-(5-nitropyridin-2-ylamino)phenoxy]benzyl}morpholin-3-one;
13). 4-Methyl-5-(4-{4-[5-(trifluoromethyl)pyridin-2-yl)amino]phenoxy}benzyl)morpholin-3-one;
14). 5-(4-{4-[Methyl(pyridin-2-yl)amino]phenoxy}benzyl)-4-methyl morpholin-3-one;
15). 4-Methyl-5-{4-[2-fluoro-4-(5-nitropyridin-2-ylamino)phenoxy]benzyl}morpholin-3-one;
16). 4-Methyl-5-{4-[2-fluoro-4-(5-(trifluoromethyl)pyridin-2-ylamino) phenoxy]benzyl}morpholin-3-one;
17). 2-[(3-Fluoro-4-{4-[(4-methyl-5-oxomorpholin-3-yl)methyl]phenoxy}phenyl)amino]nicotinonitrile;
18). 2-[(4-{4-[(4-methyl-5-oxomorpholin-3-yl)methyl]phenoxy}phenyl)amino]nicotinonitrile;
19). 4-Methyl-5-{4-[2-fluoro-4-(pyridin-2-ylamino)phenoxy]benzyl}morpholin-3-one;
20). 5-(4-{2-Fluoro-4-[methyl(5-nitropyridin-2-yl)amino]phenoxy}benzyl)-4-methyl morpholin-3-one;
21). 5-(4-{2-Fluoro-4-[methyl(3-nitropyridin-2-yl)amino]phenoxy}benzyl)-4-methyl morpholin-3-one;
22). 5-(4-{4-[Ethyl(5-nitropyridin-2-yl)amino]phenoxy}benzyl)-4-methyl morpholin-3-one;
23). 5-(4-{2-Fluoro-4-[ethyl(5-nitropyridin-2-yl)amino]phenoxy}benzyl)-4-methyl morpholin-3-one;
24). 5-(4-{4-[Methyl(3-nitropyridin-2-yl)amino]phenoxy}benzyl)-4-methyl morpholin-3-one;
25). 5-(4-{2-Fluoro-4-[ethyl(5-(trifluoromethyl)pyridin-2-yl)amino]phenoxy}benzyl)-4-methyl morpholin-3-one;
26). 5-(4-{4-[Ethyl(pyridin-2-yl)amino]phenoxy}benzyl)-4-methyl morpholin-3-one;
27). 5-(4-{4-[Methyl(5-nitropyridin-2-yl)amino]phenoxy}benzyl)-4-methyl morpholin-3-one;
28). 5-{4-[4-(5-Nitropyridin-2-ylamino)phenoxy]benzyl}-4-ethyl morpholin-3-one;
29). 2-[Methyl(4-{4-[(4-methyl-5-oxomorpholin-3-yl)methyl]phenoxy}phenyl)amino]nicotinonitrile;
30). -5-{4-[2-Fluoro-4-(3-nitropyridin-2-ylamino)phenoxy]benzyl}-4-ethyl morpholin-3-one;
31). 2-[(3-Fluoro-4-{4-[(4-methyl-5-oxomorpholin-3-yl)methyl]phenoxy}phenyl)(methyl)amino]nicotinonitrile;
32). 5-{4-[4-(3-Nitropyridin-2-ylamino)phenoxy]benzyl}-4-ethyl morpholin-3-one;
33). 5-{4-[2-Fluoro-4-(pyridin-2-ylamino)phenoxy]benzyl}-4-ethyl morpholin-3-one;
34). 2-[(4-{4-[(4-Ethyl-5-oxomorpholin-3-yl)methyl]phenoxy}phenyl)amino]nicotinonitrile;
35). 5-{4-[4-(Pyridin-2-ylamino)phenoxy]benzyl}-4-ethyl morpholin-3-one;
36). 5-{4-[2-Fluoro-4-(5-nitropyridin-2-ylamino)phenoxy]benzyl}-4-ethyl morpholin-3-one;
37). 2-[(3-Fluoro-4-{4-[(4-ethyl-5-oxomorpholin-3-yl)methyl]phenoxy}phenyl)amino]nicotinonitrile;
38). N-Ethyl-3-nitro-N-{4-[4-{(4-methylmorpholin-3-yl)methyl}phenoxy]phenyl}-pyridin-2-amine;
39). 2-[(4-{4-[(4-Ethyl-5-oxomorpholin-3-yl)methyl]phenoxy}phenyl)amino]nicotinonitrile;
40). 5-(4-{2-Fluoro-4-[(5-(trifluoromethyl)pyridin-2-yl)amino]phenoxy}benzyl)-4-ethyl morpholin-3-one;
41). 5-(4-{2-Fluoro-4-[methyl(3-nitropyridin-2-yl)amino]phenoxy}benzyl)-4-ethyl morpholin-3-one;
42). 5-(4-{2-Fluoro-4-[methyl(5-nitropyridin-2-yl)amino]phenoxy}benzyl)-4-ethyl morpholin-3-one;
43). 5-(4-{2-Fluoro-4-[ethyl(5-nitropyridin-2-yl)amino]phenoxy}benzyl)-4-ethyl morpholin-3-one;
44). 5-(4-{4-[(5-(Trifluoromethyl)pyridin-2-yl)amino]phenoxy}benzyl)-4-ethyl morpholin-3-one;
45). 2-[(4-{4-[(4-Ethyl-5-oxomorpholin-3-yl)methyl]phenoxy}phenyl) (ethyl)amino]nicotinonitrile;
46). 2-[(3-Fluoro-4-{4-[(4-ethyl-5-oxomorpholin-3-yl)methyl]phenoxy}phenyl) (methyl)amino]nicotinonitrile;
47). 2-[(3-Fluoro-4-{4-[(4-ethyl-5-oxomorpholin-3-yl)methyl]phenoxy}phenyl) (ethyl)amino]nicotinonitrile;
48). 5-(4-{2-Fluoro-4-[ethyl(5-(trifluoromethyl)pyridin-2-yl)amino]phenoxy}benzyl)-4-ethyl morpholin-3-one;
49). 5-(4-{2-Fluoro-4-[methyl(pyridin-2-yl)amino]phenoxy}benzyl)-4-ethyl morpholin-3-one;
50). 5-(4-{4-[Methyl(5-(trifluoromethyl)pyridin-2-yl)amino]phenoxy}benzyl)-4-ethyl morpholin-3-one;
51). 5-(4-{4-[Methyl(3-nitropyridin-2-yl)amino]phenoxy}benzyl)-4-ethyl morpholin-3-one;
52). 5-(4-{4-[Ethyl(3-nitropyridin-2-yl)amino]phenoxy}benzyl)-4-ethyl morpholin-3-one;
53). 5-(4-{4-[Methyl(5-(trifluoromethyl)pyridin-2-yl)amino]phenoxy}benzyl)-4-methyl morpholin-3-one;
54). 5-(4-{4-[Ethyl(5-(trifluoromethyl)pyridin-2-yl)amino]phenoxy}benzyl)-4-methyl morpholin-3-one;
55). 5-Nitro N-{4-[4-{(4-methylmorpholin-3-yl)methyl}phenoxy]phenyl}-pyridin-2-amine;
56). N-ethyl-5-nitro-N-{4-[4-{(4-methylmorpholin-3-yl)methyl}phenoxy]phenyl}-pyridin-2-amine and
57). 3-Nitro-N-{4-[4-{(4-methylmorpholin-3-yl)methyl}phenoxy]phenyl}-pyridin-2-amine.

3. The compound as claimed in claim 1, wherein the said pharmaceutically acceptable salt is selected from the group consisting of hydrochloride, hydrobromide, sodium, potassium or magnesium.

4. A pharmaceutical composition, which comprises of a pharmaceutically effective amount of novel heterocyclic analogs of biphenyl ethers of formula (I), 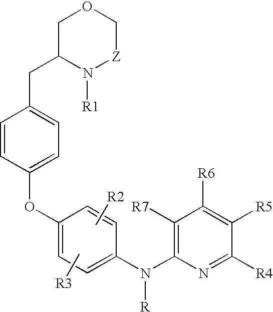
as defined in claim 1 and a pharmaceutically acceptable carrier, diluent, excipient or solvate.

5. A pharmaceutical composition as claimed in claim 1, in the form of a tablet, capsule, powder, syrup, solution, aerosol or suspension.

6. A pharmaceutical composition as claimed in claim 1, wherein the amount of the compound of claim 1 in the composition is less than 60% by weight.

7. A method for reducing blood glucose, free fatty acids, cholesterol, triglycerides levels in plasma comprising administration of an effective amount of a compound of formula (I) as defined in claim 1, to a patient in need thereof.

8. A method for treating obesity, autoimmune diseases, inflammation, immunological diseases, and cancer disease comprising administration of an effective amount of a compound of formula (I) as defined in claim 1, to a patient in need thereof.

9. A method for treating a disorder associated with insulin resistance comprising administration of an effective amount of a compound of formula (D) as defined in claim 1, to a patient in need thereof.

10. A method for reducing blood glucose levels in the plasma without adipogenic potential comprising administration of an effective amount of a compound as claimed in claim 1, to a mammal in need thereof.

11. A method for reducing blood glucose levels in the plasma without adipogenic potential comprising administration of an effective amount of a compound as claimed in claim 2, to a mammal in need thereof.
